(19) United States
(12) Patent Application Publication
(10) Pub. No.: US 2024/0238199 A1
(43) Pub. Date: Jul. 18, 2024

(54) STABLE LIQUID PHARMACEUTICAL COMPOSITIONS HAVING HIGH DRUG LOADINGS OF MEDIUM CHAIN TRIGLYCERIDES AND METHODS RELATED THERETO

(71) Applicant: CERECIN INC., Denver, CO (US)

(72) Inventors: Aikun Julie LIU, Geelong (AU); Thomas KAASGAARD, Clayton (AU); Murali NAIR C K BALACHANDRAN, Singapore (SG); Samuel T. HENDERSON, Golden, CO (US); Benjamin BOYD, Clayton (AU)

(21) Appl. No.: 18/563,204

(22) PCT Filed: May 25, 2022
(86) PCT No.: PCT/US2022/030819

§ 371 Date: Nov. 21, 2023

Related U.S. Application Data
(60) Provisional application No. 63/192,826, filed on May 25, 2021.

(51) Int. Cl. A61K 9/107; A61K 31/23; A61K 47/14; A61K 47/24; A61K 47/44; A61K 47/46

(52) U.S. Cl. CPC A61K 9/1075; A61K 31/23; A61K 47/14; A61K 47/24; A61K 47/44; A61K 47/46

(57) ABSTRACT

This invention relates to high drug load compositions of medium chain triglycerides (MCT), and to methods for treatment with such compositions at amounts effective to elevate ketone body concentrations so as to treat conditions associated with reduced neuronal metabolism, for example Alzheimer's disease.

### FIELD OF THE INVENTION

This disclosure relates to liquid pharmaceutical compositions comprising high drug loadings of medium chain triglycerides, as well as methods of making and methods of using such compositions.

### BACKGROUND OF THE INVENTION

Medium Chain Triglycerides (MCTs) are comprised of fatty acids with chain length between 5-12 carbons. MCTs have been researched extensively and have known nutritional and pharmaceutical uses. MCTs having melting points which are liquid at room temperature. Further, MCTs are relatively small and, once hydrolyzed, the fatty acids formed from MCTs are ionizable at physiological pH, and hence are generally soluble in aqueous solutions.

When intended to be used as a pharmaceutical composition, it is often desirable to prepare compositions of active ingredients as a ready-to-use, preservative-free liquid dosage form at room temperature. However, it can be quite difficult to achieve long term stability, both physico-chemical and microbiological stability.

As such, there is a need in the art for ready-to-use, preservative-free liquid dosage form compositions of MCTs, particularly with sufficient long term stability and at active ingredient to excipient levels (herein referred to as drug loading) sufficiently high for pharmaceutical use.

### SUMMARY OF THE INVENTION

In one aspect, the disclosure relates to a liquid pharmaceutical composition comprising at least about 30% by weight of the total composition of caprylic triglyceride, and one or more emulsion forming excipients present at a concentration sufficient to form stable emulsion for at least one month under ambient conditions. In some aspects, the caprylic triglyceride is present in an amount of between about 30% and about 60% by weight of the total composition. In some aspects, the purity of the caprylic triglyceride is at least 95%.

In some aspects, the one or more emulsion forming excipients are selected from the group consisting of lecithin (e.g., Phospholipon 90G), hydrogenated castor oils including Polyoxyl 40 castor oil (e.g., Kolliphor RH40), caprylate esters, sodium oleate, glycerol, citric acid esters of monoglycerides and diglycerides (e.g., Citrem), monoglycerides and diglycerides of fatty acids including Propylene Glycol Monocaprylate (e.g. Capmul PG-8), and combinations thereof.

In some aspects, the one or more emulsion forming excipients are selected from the group consisting of lecithin, Kolliphor RH40, caprylate ester emulsifiers, and combinations thereof. In some aspects, the one or more emulsion forming excipients are selected from the group consisting of lecithin, sodium oleate, glycerol, and combinations thereof. In some aspects, the one or more emulsion forming excipients are selected from the group consisting of Citrem, monoglycerides and diglycerides of fatty acids, and combinations thereof.

In some aspects, the one or more emulsion forming excipients are present in an amount of between about 1% and about 10% by weight of the total composition, preferably in an amount of between about 1% and about 8% by weight of the total composition.

In some aspects, there are at least two emulsion forming excipients present in the composition, and at least one of the emulsion forming excipients is present in an amount of at least 2.0% by weight of the total composition. In some aspects, the at least two emulsion forming excipients are present at a 1:1 to 2:1 ratio, relative to one another.

In some aspects, the liquid pharmaceutical composition of the disclosure forms an emulsion that is stable for at least about one month at ambient conditions. In some aspects, the stable emulsions exhibits an average particle diameter of less than 0.5 μm for at least one month at ambient conditions, preferrable less than 0.3 μm for at least one month at ambient conditions, preferrable less than 0.2 μm for at least one month at ambient conditions. In other aspects, the emulsions may have a mean particle diameter of less than about 1000 nm, but greater than about 100 nm, e.g. between about 100 nm and 500 nm, between about 200 nm and about 300 nm, between about 160 nm and about 190 nm, etc.

In some aspects, the liquid pharmaceutical composition of the disclosure further comprises an oil soluble flavouring agent.

In yet other aspects, the disclosure relates to methods of treating a disease or disorder associated with reduced cognitive function in a subject in need thereof, the method comprising administering to the subject a liquid pharmaceutical composition of the disclosure in an amount effective to elevate ketone body concentrations in said subject to thereby treat said disease or disorder. In certain embodiments, the disease or disorder associated with reduced cognitive function is selected from Alzheimer's disease and Age-Associated Memory Impairment.

While multiple embodiments are disclosed, still other embodiments of the present disclosure will become apparent to those skilled in the art from the following detailed description, which shows and describes illustrative embodiments of the disclosure. As will be realized, the invention is capable of modifications in various aspects, all without departing from the spirit and scope of the present disclosure. Accordingly, the detailed descriptions are to be regarded as illustrative in nature and not restrictive.

### BRIEF DESCRIPTION OF THE DRAWINGS

FIG. 1 illustrates an exemplary method for preparation of liquid pharmaceutical compositions of the disclosure.

FIG. 2 illustrates exemplary components, concentrations and compositions of liquid pharmaceutical compositions, in accordance with embodiments of the disclosure.

FIG. 3 illustrates a region of optimized performing compositions of liquid pharmaceutical compositions of FIG. 2, in accordance with embodiments of the disclosure.

FIG. 4 illustrates performance of compositions of liquid pharmaceutical compositions of FIG. 2, in accordance with embodiments of the disclosure.

FIG. 5 illustrates performance of compositions of liquid pharmaceutical compositions of FIG. 2, in accordance with embodiments of the disclosure.

FIG. 6 illustrates performance of compositions of liquid pharmaceutical compositions of FIG. 2, in accordance with embodiments of the disclosure.

FIG. 7. illustrates exemplary particle size distributions for different Citrem formulations, in accordance with embodiments of the disclosure.

FIG. 8 illustrates stability results for Oleate formulations, in accordance with embodiments of the disclosure.

FIG. 9 illustrates visual stability results for Oleate formulations, in accordance with embodiments of the disclosure.

FIG. 10 illustrates stability results for Oleate formulations, in accordance with embodiments of the disclosure.

FIG. 11 illustrates stability results for Oleate formulations, in accordance with embodiments of the disclosure.

FIG. 12 illustrates visual stability results for Oleate formulations, in accordance with embodiments of the disclosure.

FIG. 13 illustrates stability results for Oleate formulations, in accordance with embodiments of the disclosure.

FIG. 14 illustrates stability results for Oleate formulations, in accordance with embodiments of the disclosure.

FIG. 15 illustrates visual stability results for Oleate formulations, in accordance with embodiments of the disclosure.

FIG. 16 illustrates stability results for Oleate formulations, in accordance with embodiments of the disclosure.

FIG. 17 illustrates stability results for Oleate formulations, in accordance with embodiments of the disclosure.

FIG. 18 illustrates visual stability results for Oleate formulations, in accordance with embodiments of the disclosure.

FIG. 19 illustrates stability results for Oleate formulations, in accordance with embodiments of the disclosure.

FIG. 20. illustrates exemplary particle size distributions for different formulations of gamma-irradiated vs retention samples, in accordance with embodiments of the disclosure.

FIGS. 21A-21J illustrate exemplary particle size distributions for lead formulations over time, in accordance with embodiments of the disclosure.

FIG. 22A illustrates pharmacokinetic parameters, including total ketone Cmax and FIG. 22B illustrates total ketone AUC for lead formulations, in accordance with embodiments of the disclosure.

### DETAILED DESCRIPTION OF THE INVENTION

By way of background, Medium Chain Triglyceride (“MCT”s) are metabolized differently from the more common Long Chain Triglycerides (LCTs). In particular, when compared to LCTs, MCTs are more readily digested to release medium chain fatty acids (MCFAs), which exhibit increased rates of portal absorption, and undergo obligate oxidation. The small size and decreased hydrophobicity of MCTs increases the rate of digestion and absorption relative to LCTs. When MCTs are ingested, they are first processed by lipases, which cleave the fatty acid chains from the glycerol backbone. Some lipases in the pre-duodenum preferentially hydrolyze MCTs over LCTs, and the released MCFAs are then partly absorbed directly by the stomach mucosa. Those MCFAs which are not absorbed in the stomach are absorbed directly into the portal vein and not packaged into lipoproteins. Since blood transports much more rapidly than lymph, MCFAs quickly arrive at the liver. In the liver MCFAs undergo obligate oxidation.

In contrast, long chain fatty acids (LCFAs) derived from normal dietary fat are re-esterified into LCTs and packaged into chylomicrons for transport in the lymph. This greatly slows the metabolism of LCTs relative to MCTs. In the fed state LCFAs undergo little oxidation in the liver, due mainly to the inhibitory effects of malonyl-CoA. When conditions favor fat storage, malonyl-CoA is produced as an intermediate in lipogenesis. Malonyl-CoA allosterically inhibits carnitine palmitoyltransferase I, and thereby inhibits LCFA transport into the mitochondria. This feedback mechanism prevents futile cycles of lipolysis and lipogenesis.

MCFAs are, to a large extent, immune to the regulations that control the oxidation of LCFAs. MCFAs enter the mitochondria without the use of carnitine palmitoyltransferase I, therefore MCFAs by-pass this regulatory step and are oxidized regardless of the metabolic state of the organism. Importantly, since MCFAs enter the liver rapidly and are quickly oxidized, large amounts of ketone bodies are readily produced from MCFAs. As such, a large oral dose of MCTs (e.g., about 20 mL to 40 mL) will result in sustained hyperketonemia.

The present disclosure generally relates to liquid pharmaceutical compositions comprising a high loading of at least one MCT, and methods of making and using such compositions. In certain embodiments, the liquid pharmaceutical compositions form a stable liquid emulsion in an aqueous use environment, e.g., in water or when administered to an aqueous use environment. In certain embodiments, the MCT is caprylic triglyceride, as described herein.

In certain embodiments, the liquid pharmaceutical formulations may be “preservative free”. In such embodiments, the formulation may form a stable liquid emulsion that maintains sterility (i.e., sufficient bio-burden reduction to allow for pharmaceutical use) for at least 1 month, at least 2 months, at least 3 months, at least 6 months, at least 12 months at ambient conditions without the use of preservatives.

In certain aspects, the pharmaceutical compositions of the disclosure are a liquid pharmaceutical composition comprising a high drug loading of an MCT, e.g., caprylic triglyceride, and one or more emulsion forming excipients present at a concentration sufficient to form an emulsion at ambient conditions. The pharmaceutical compositions may comprise the components in amounts as described herein. In some embodiments, the composition may form a stable liquid emulsion at ambient conditions, e.g., for at least 1 day, at least 1 month, at least 2 months, at least 3 months, at least 6 months, at least 12 months, etc. The emulsions of the disclosure may generally be formed by high shear mixing or “high pressure homogenization, as is understood in the art.

As described herein, the pharmaceutical compositions of the disclosure may form a stable liquid emulsion at ambient conditions. An emulsion refers to a composition which, when diluted with water or other aqueous medium and gently mixed, yields a stable oil/water emulsion with a mean particle size of less than about 1 μm, but greater than about 100 nm, (i.e., 0.1-1 μm) and which is generally polydisperse. Such an emulsion is stable, meaning there is no visibly detectable phase separation and that there is no visibly detectable crystallization.

In some aspects, the pharmaceutical composition of the disclosure forms an emulsion that is stable for at least about one month at ambient conditions. In some aspects, the stable emulsions exhibits an average particle diameter of less than 0.5 μm for at least one month at ambient conditions, preferrable less than 0.3 μm for at least one month at ambient conditions, preferrable less than 0.2 μm for at least one month at ambient conditions. In other aspects, the emulsions may have a mean particle diameter of less than about 1000 nm, but greater than about 100 nm, e.g. between about 100 nm and 500 nm, between about 200 nm and about 300 nm, between about 160 nm and about 190 nm, etc.

As discussed above, the pharmaceutical compositions of the disclosure form stable emulsions in an aqueous use environment, e.g., in water, pharmaceutically suitable aqueous solution, or when administered in vivo. By way of example, the emulsions may be stable at ambient conditions for at least about 24 hours, for at least 1 day, at least 1 month, at least 2 months, at least 3 months, at least 6 months, at least 12 months, etc. In certain embodiments, the emulsion formed does not phase separate for the duration of stability. In certain embodiments, the emulsions may have a mean particle diameter of less than about 1 μm, but greater than about 100 nm, (i.e., 0.1-1 μm).

In certain embodiments, the emulsion formed may be stable at stomach pH, e.g., at a pH of about 1 to about 3, about 1.2 to 2.9, etc. In certain embodiments, the emulsion formed may be stable at intestinal and/or colon pH, e.g., at a pH of about 5 to about 7, about 5.5 to about 6.9, etc. In certain embodiments, the emulsion formed may begin to break down or phase separate at stomach pH after about ½ to about 1 hour, but does not release the encapsulated MCT until intestinal or colon pH. In this regard, without intending to be limited by theory, in-vitro digestion assays indicate that encapsulated MCT is released from emulsion at intestinal and/or colon pH, which is the primary location of lipid digestion enzymes. In accordance with certain aspects of the disclosure, preferential release of MCT in the intestines and/or colon rather than the stomach may increase bioavailability of the MCT given the location of lipid digestion enzymes in these areas.

In certain aspects of the disclosure, the pharmaceutical compositions provide for preferential release of the high drug loading of MCT in the lower gastrointestinal tract of a user. Without intending to be limited by theory, preferential release of MCT in the lower gastrointestinal tract, including the colon may provide reduced stomach upset and related adverse events as compared to standard administration of non-formulated MCT oil. Further, the improved bioavailability of the MCT may generally lead to increased ketone body production in vivo, as compared to standard administration of non-formulated MCT oil.

In certain embodiments, the pharmaceutical compositions may include a high drug load of at least one MCT, such as caprylic triglyceride, of at least about 20% of the total composition, at least about 25% of the total composition, at least about 30% by weight of the total composition, at least about 40% by weight of the total composition, about 30% by weight of the total composition to about 65% by weight of the total composition, about 30% by weight of the total composition to about 60% by weight of the total composition, about 40% by weight of the total composition to about 50% by weight of the total composition, about 40% by weight of the total composition to about 45% by weight of the total composition, etc.

As used herein, unless other specified, “% by weight” refers to “% by weight of the total composition”.

In certain aspects of the disclosure, MCTs refer to any glycerol molecule ester-linked to three fatty acid molecules, each fatty acid molecule having a carbon chain of 5-12 carbons. In certain embodiments, the pharmaceutical compositions may comprise an MCT represented by the following general formula:

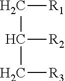

wherein R₁, R₂ and R₃ are fatty acids having 5-12 carbons in the carbon backbone esterified to the a glycerol backbone.

The MCTs of the disclosure may be prepared by any process known in the art, such as direct esterification, rearrangement, fractionation, transesterification, or the like. Sources of the MCT include any suitable source, semi-synthetic, synthetic or natural. Examples of natural sources of MCT include plant sources such as coconuts and coconut oil, palm kernels and palm kernel oils, and animal sources such as milk from any of a variety of species, e.g., goats. For example, the lipids may be prepared by the rearrangement of a vegetable oil such as coconut oil. The length and distribution of the chain length may vary depending on the source oil. For example, MCT containing 1-10% C6, 30-60% C8, 30-60% C10, 1-10% C10 are commonly derived from palm and coconut oils.

In accordance with certain embodiments of the disclosure, the pharmaceutical compositions of the disclosure may comprise MCTs that have greater than about 95% C8 at R₁, R₂ and R₃, and are herein referred to herein as caprylic triglyceride (“CT”). Exemplary sources of CT include MIGLYOL® 808 or NEOBEE® 895. In certain aspects, CT may be obtained from coconut or palm kernel oil, made by semi-synthetic esterification of octanoic acid to glycerin, etc.

In other embodiments, the pharmaceutical compositions may comprise MCTs wherein R₁, R₂, and R₃ are fatty acids containing a six-carbon backbone (tri-C6:0). Tri-C6:0 MCT are absorbed very rapidly by the gastrointestinal tract in a number of animal model systems. The high rate of absorption results in rapid perfusion of the liver, and a potent ketogenic response. In another embodiment, the pharmaceutical compositions may comprise MCTs wherein R₁, R₂, and R₃ are fatty acids containing an eight-carbon backbone (tri-C8:0). In another embodiment, the pharmaceutical compositions may comprise MCTs wherein R₁, R₂, and R₃ are fatty acids containing a ten-carbon backbone (tri-C10:0). In another embodiment, the pharmaceutical compositions may comprise MCTs wherein R₁, R₂, and R₃ are a mixture of C8:0 and C10:0 fatty acids. In another embodiment, the pharmaceutical compositions may comprise MCTs wherein R₁, R₂ and R₃ are a mixture of C6:0, C8:0, C10:0, and C12:0 fatty acids. In another embodiment, the pharmaceutical compositions may comprise MCTs wherein greater than 95% of R₁, R₂ and R₃ are 8 carbons in length. In yet another embodiment, the pharmaceutical compositions may comprise MCTs wherein the R₁, R₂, and R₃ carbon chains are 6-carbon or 10-carbon chains. In another embodiment, the pharmaceutical compositions may comprise MCTs wherein about 50% of R₁, R₂ and R₃ are 8 carbons in length and about 50% of R₁, R₂ and R₃ 10 carbons in length. In one embodiment, the pharmaceutical compositions may comprise MCTs wherein R₁, R₂ and R₃ are 6, 8, 10 or 12 carbon chain length, or mixtures thereof.

In certain aspects, the liquid pharmaceutical formulations of the disclosure include one or more emulsion forming excipients. In certain embodiments, the one or more emulsion forming excipients may be any emulsifier capable of forming an emulsion with MCT oil. By way of example, lecithin (e.g., Phospholipon 90G), hydrogenated castor oils including Polyoxyl 40 castor oil (e.g., Kolliphor RH40), caprylate esters, sodium oleate, glycerol, citric acid esters of monoglycerides and diglycerides (e.g., Citrem), monoglycerides and diglycerides of fatty acids including Propylene Glycol Monocaprylate (e.g. Capmul PG-8), and combinations thereof. The emulsion forming excipient(s) may be present in amounts sufficient to provide desired emulsion formation. For example, in certain embodiments, the emulsion forming excipient may be present in an amount of between about 1% and about 10%, between about 1% and about 8% by weight of the total composition, between about 1.3% and about 10%, etc., by weight of the total composition.

In some aspects, there are at least two emulsion forming excipients present in the composition, and at least one of the emulsion forming excipients is present in an amount of at least 2.0% by weight of the total composition. In some aspects, the at least two emulsion forming excipients are present at a 1:1 to 2:1 ratio, relative to one another.

In certain embodiments, the emulsion forming excipients may include various combinations of lecithin, Kolliphor RH40, and caprylate ester emulsifiers, and optionally glycerol. In other embodiments, the emulsion forming excipients may include various combinations of lecithin, sodium oleate, and optionally glycerol. In yet other embodiments, the emulsion forming excipients may include Citrem alone or in combination with monoglycerides and diglycerides of fatty acids.

In certain embodiments, the liquid pharmaceutical formulations of the disclosure may optionally include one or more flavouring or sweetener agents. In certain embodiments, the flavouring or sweetening agent may be present in an amount of between 0.025% to 0.3%, between 0.15% to 0.3%, 0.5% for sweetening agent, 0.3% for flavouring agent, etc. by weight of the total composition. In certain embodiments, sucralose, stevia or similar sweetening agents may be used, with sucralose being preferred. In certain embodiments, vanilla, mango, berry, or similar flavouring agents may be used, with vanilla being preferred. In some embodiments, the flavouring or sweetening agents may be oil soluble.

By way of non-limiting example, Table 1 below provides exemplary liquid pharmaceutical formulation attributes and properties.

TABLE 1

| Product Attribute | | Property |
|---|---|---|
| Exemplary Indication(s) | | Migraine, Headache, Alzheimer, Neuroinflammation |
| Patient Population | | Young Adults, Adults, Geriatric |
| Dosage Form | | Oil in liquid emulsion, single-dose |
| | | Ready to drink |
| | | 30 mL light protected container |
| Particle size of the emulsion | | 0.35-1.2 um |
| Potency and Impurity | | 90-110%, total impurity less than 5% |
| pH | | Neutral or near Neutral (pH 6-pH 8) |
| Dosing Interval | | Once daily, Twice daily, Three times daily |
| Dose | Primary | 50 g/100 mL (50% w/v MCT) |
| Strength(s) | strengths | |
| | Max/min | To allow for doses of 5 g to 20 g as a single dose |
| | dosing vol. | |
| Dosing | Time and | 30′ after completion of a meal |
| Regimen | duration | |

| | |
|---|---|
| Formulation Ingredients, | Phospholipon 90G, Kolliphor RH40, Citrem, |
| alone and in selected | Monoglycerides, Capmul PG-8, Sodium Oleate, |
| combination | Glycerol, flavouring/sweetening agent(s) |

By way of non-limiting example, suitable lecithin useful as an emulsion forming excipient of the disclosure may be derived from any suitable source, e.g., egg or soy. By way of non-limiting example, suitable lecithin may be selected from Soy PC, 95%, Avanti Number 441601; Egg PC, 95%, Avanti Number 131601; etc.

Any suitable monoglyceride or diglyceride of fatty acids may be used as an emulsion forming agent of the disclosure, e.g., citric acid esters of mono and diglycerides of fatty acids (Citrem) E472C; mono and diglycerides of fatty acids E471; etc.

Any suitable method for making the pharmaceutical compositions of the disclosure may be used. In certain aspects of the disclosure, it has been found that reproducibility and emulsion stability may be controlled by modifying manufacturing process as illustrated in the examples herein.

In certain aspects, the disclosure relates to methods of treating a disease or disorder associated with reduced cognitive function in a subject in need thereof, the method comprising administering to the subject a pharmaceutical composition of the disclosure in an amount effective to elevate ketone body concentrations in said subject to thereby treat said disease or disorder. In certain embodiments, the pharmaceutical composition of the disclosure may be administered outside of the context of a ketogenic diet. For instance, in the context of the present disclosure, carbohydrates may be consumed at the same time as pharmaceutical compositions disclosed herein.

In accordance with certain aspects of the disclosure, diseases and disorders associated with reduced cognitive function include Age-Associated Memory Impairment (AAMI), Alzheimer's Disease (AD), Parkinson's Disease, Friedreich's Ataxia (FRDA), GLUT1-deficient Epilepsy, Leprechaunism, and Rabson-Mendenhall Syndrome, Coronary Arterial Bypass Graft (CABG) dementia, anesthesia-induced memory loss, Huntington's Disease, migraine and related headaches, and many others.

In another embodiment, the patient has or is at risk of developing disease-related reduced cognitive function caused by reduced neuronal metabolism, for example, reduced cognitive function associated with Alzheimer's Disease (AD), Parkinson's Disease, Friedreich's Ataxia (FRDA), GLUT1-deficient Epilepsy, Leprechaunism, and Rabson-Mendenhall Syndrome, Coronary Arterial Bypass Graft (CABG) dementia, anesthesia-induced memory loss, Huntington's Disease, and many others.

As used herein, reduced neuronal metabolism refers to all possible mechanisms that could lead to a reduction in neuronal metabolism. Such mechanisms include, but are not limited to mitochondrial dysfunction, free radical attack, generation of reactive oxygen species (ROS), ROS-induced neuronal apoptosis, defective glucose transport or glycolysis, imbalance in membrane ionic potential, dysfunction in calcium flux, and the like.

According to the present invention, high blood ketone levels will provide an energy source for brain cells that have compromised glucose metabolism, leading to improved performance in cognitive function. As used herein, “subject” and “patient” are used interchangeably, and refer to any mammal, including humans that may benefit from treatment of disease and conditions associated with or resulting from reduced neuronal metabolism.

“Effective amount” refers to an amount of a compound, material, or pharmaceutical composition, as described herein that is effective to achieve a particular biological result. Effectiveness for treatment of the aforementioned conditions may be assessed by improved results from at least one neuropsychological test. These neuropsychological tests are known in the art and include Clinical Global Impression of Change (CGIC), Rey Auditory Verbal Learning Test (RAVLT), First-Last Names Association Test (FLN), Telephone Dialing Test (TDT), Memory Assessment Clinics Self-Rating Scale (MAC-S), Symbol Digit Coding (SDC), SDC Delayed Recall Task (DRT), Divided Attention Test (DAT), Visual Sequence Comparison (VSC), DAT Dual Task (DAT Dual), Mini-Mental State Examination (MMSE), and Geriatric Depression Scale (GDS), among others.

The term “cognitive function” refers to the special, normal, or proper physiologic activity of the brain, including, without limitation, at least one of the following: mental stability, memory/recall abilities, problem solving abilities, reasoning abilities, thinking abilities, judging abilities, capacity for learning, perception, intuition, attention, and awareness. “Enhanced cognitive function” or “improved cognitive function” refers to any improvement in the special, normal, or proper physiologic activity of the brain, including, without limitation, at least one of the following: mental stability, memory/recall abilities, problem solving abilities, reasoning abilities, thinking abilities, judging abilities, capacity for learning, perception, intuition, attention, and awareness, as measured by any means suitable in the art. “Reduced cognitive function” or “impaired cognitive function” refers to any decline in the special, normal, or proper physiologic activity of the brain.

In another embodiment, the methods of the present invention further comprise determination of the patients' genotype or particular alleles. In one embodiment, the patient's alleles of the apolipoprotein E gene are determined. It has been found that non-E4 carriers performed better than those with the E4 allele when elevated ketone body levels were induced with MCT. Also, those with the E4 allele had higher fasting ketone body levels and the levels continued to rise at the two hour time interval. Therefore, E4 carriers may require higher ketone levels or agents that increase the ability to use the ketone bodies that are present.

In one embodiment, the pharmaceutical compositions of the disclosure are administered orally. Therapeutically effective amounts of the therapeutic agents can be any amount or dose sufficient to bring about the desired effect and depend, in part, on the severity and stage of the condition, the size and condition of the patient, as well as other factors readily known to those skilled in the art. The dosages can be given as a single dose, or as several doses, for example, divided over the course of several weeks, as discussed elsewhere herein.

The pharmaceutical compositions of the disclosure, in one embodiment, are administered in a dosage required to increase blood ketone bodies to a level required to treat and/or prevent the occurrence of any disease- or age-associated cognitive decline, such as AD, AAMI, and the like. Appropriate dosages may be determined by one of skill in the art.

In one embodiment, oral administration of a pharmaceutical composition of the disclosure results in hyperketonemia. Hyperketonemia, in one embodiment, results in ketone bodies being utilized for energy in the brain even in the presence of glucose. Additionally, hyperketonemia results in a substantial (39%) increase in cerebral blood flow (Hasselbalch, S. G., et al., Changes in cerebral blood flow and carbohydrate metabolism during acute hyperketonemia, Am J Physiol, 1996, 270:E746-51). Hyperketonemia has been reported to reduce cognitive dysfunction associated with systemic hypoglycemia in normal humans (Veneman, T., et al., Effect of hyperketonemia and hyperlacticacidemia on symptoms, cognitive dysfunction, and counterregulatory hormone responses during hypoglycemia in normal humans, Diabetes, 1994, 43:1311-7). Please note that systemic hypoglycemia is distinct from the local defects in glucose metabolism that occur in any disease- or age-associated cognitive decline, such as AD, AAMI, and the like.

Administration can be on an as-needed or as-desired basis, for example, once-monthly, once-weekly, daily, or more than once daily. Similarly, administration can be every other day, week, or month, every third day, week, or month, every fourth day, week, or month, and the like. Administration can be multiple times per day. When utilized as a supplement to ordinary dietetic requirements, the composition may be administered directly to the patient or otherwise contacted with or admixed with daily feed or food.

The pharmaceutical compositions provided herein are, in one embodiment, intended for “long term” consumption, sometimes referred to herein as for ‘extended’ periods. “Long term” administration as used herein generally refers to periods in excess of one month. Periods of longer than two, three, or four months comprise one embodiment of the instant invention. Also included are embodiments comprising more extended periods that include longer than 5, 6, 7, 8, 9, or 10 months. Periods in excess of 11 months or 1 year are also included. Longer terms use extending over 1, 2, 3 or more years are also contemplated herein. “Regular basis” as used herein refers to at least weekly, dosing with or consumption of the compositions. More frequent dosing or consumption, such as twice or thrice weekly are included. Also included are regimens that comprise at least once daily consumption. The skilled artisan will appreciate that the blood level of ketone bodies, or a specific ketone body, achieved may be a valuable measure of dosing frequency. Any frequency, regardless of whether expressly exemplified herein, that allows maintenance of a blood level of the measured compound within acceptable ranges can be considered useful herein. The skilled artisan will appreciate that dosing frequency will be a function of the composition that is being consumed or administered, and some compositions may require more or less frequent administration to maintain a desired blood level of the measured compound (e.g., a ketone body).

Administration can be carried out on a regular basis, for example, as part of a treatment regimen in the patient. A treatment regimen may comprise causing the regular ingestion by the patient of a pharmaceutical composition of the disclosure in an amount effective to enhance cognitive function, memory, and behavior in the patient. Regular ingestion can be once a day, or two, three, four, or more times per day, on a daily or weekly basis. Similarly, regular administration can be every other day or week, every third day or week, every fourth day or week, every fifth day or week, or every sixth day or week, and in such a regimen, administration can be multiple times per day. The goal of regular administration is to provide the patient with optimal dose of a pharmaceutical composition of the disclosure, as exemplified herein.

Dosages of the inventive compositions, such as, for example, those comprising MCT, may be administered in an effective in an effective amount to increase the cognitive ability of patients afflicted with diseases of reduced neuronal metabolism, such as in patients with any disease- or age-associated cognitive decline, such as, AD, AAMI, and the like.

In one embodiment, the inventive compositions result in elevating ketone concentrations in the body, and in this embodiment, the compositions are administered in an amount that is effective to induce hyperketonemia. In one embodiment, hyperketonemia results in ketone bodies being utilized for energy in the brain.

In one embodiment, the composition increases the circulating concentration of at least one type of ketone body in the mammal or patient. In one embodiment, the circulating ketone body is D-beta-hydroxybutyrate. The amount of circulating ketone body can be measured at a number of times post administration, and in one embodiment, is measured at a time predicted to be near the peak concentration in the blood, but can also be measured before or after the predicted peak blood concentration level. Measured amounts at these off-peak times are then optionally adjusted to reflect the predicted level at the predicted peak time. In one embodiment, the predicted peak time is at about two hours. Peak circulating blood level and timing can vary depending on factors known to those of skill in the art, including individual digestive rates, co-ingestion or pre- or post-ingestion of foods, drinks, etc., as known to one of skill in the art. In one embodiment, the peak blood level reached of D-beta-hydroxybutyrate is between about 0.05 millimolar (mM) to about 50 mM. Another way to determine whether blood levels of D-beta-hydroxybutyrate are raised to about 0.05 to about 50 mM is by measurement of D-beta-hydroxybutyrate urinary excretion a range in the range of about 5 mg/dL to about 160 mg/dL. In other embodiments, the peak blood level is raised to about 0.1 to about 50 mM, from about 0.1 to about 20 mM, from about 0.1 to about 10 mM, to about 0.1 to about 5 mM, more preferably raised to about 0.15 to about 2 mM, from about 0.15 to about 0.3 mM, and from about 0.2 to about 5 mM, although variations will necessarily occur depending on the formulation and host, for example, as discussed above. In other embodiments, the peak blood level reached of D-beta-hydroxybutyrate will be at least about 0.05 mM, at least about 0.1 mM, at least about 0.15 mM, at least about 0.2 mM, at least about 0.5 mM, at least about 1 mM, at least about 1.5 mM, at least about 2 mM, at least about 2.5 mM, at least about 3 mM, at least about 4 mM, at least about 5 mM, at least about 10 mM, at least about 15 mM, at least about 20 mM, at least about 30 mM, at least about 40 mM, and at least about 50 mM.

Effective amount of dosages of compounds for the inventive compositions, i.e., compounds capable of elevating ketone body concentrations in an amount effective for the treatment of or prevention of loss of cognitive function caused by reduced neuronal metabolism will be apparent to those skilled in the art. As discussed herein above, such effective amounts can be determined in light of disclosed blood ketone levels. Where the compound capable of elevating ketone body concentrations is MCT, the MCT dose, in one embodiment, is in the range of about 0.05 g/kg/day to about 10 g/kg/day of MCT. In other embodiments, the dose will be in the range of about 0.25 g/kg/day to about 5 g/kg/day of MCT. In other embodiments, the dose will be in the range of about 0.5 g/kg/day to about 2 g/kg/day of MCT. In other embodiments, the dose will be in the range of about 0.1 g/kg/day to about 2 g/kg/day. In other embodiments, the dose of MCT is at least about 0.05 g/kg/day, at least about 0.1 g/kg/day, at least about 0.15 g/kg/day, at least about 0.2 g/kg/day, at least about 0.5 g/kg/day, at least about 1 g/kg/day, at least about 1.5 g/kg/day, at least about 2 g/kg/day, at least about 2.5 g/kg/day, at least about 3 g/kg/day, at least about 4 g/kg/day, at least about 5 g/kg/day, at least about 10 g/kg/day, at least about 15 g/kg/day, at least about 20 g/kg/day, at least about 30 g/kg/day, at least about 40 g/kg/day, and at least about 50 g/kg/day.

As described herein, the present compositions are provided as a liquid formulation for administration to a subject in need thereof. The compositions may be advantageously combined and/or used in combination with other therapeutic or prophylactic agents, different from the disclosed MCT compounds. In many instances, administration in conjunction with the subject compositions enhances the efficacy of such agents. For example, the compounds may be advantageously used in conjunction with antioxidants, compounds that enhance the efficiency of glucose utilization, and mixtures thereof.

The daily dose of MCT can also be measured in terms of grams of MCT per kg of body weight (BW) of the mammal. The daily dose of MCT can range from about 0.01 g/kg to about 10.0 g/kg BW of the mammal. Preferably, the daily dose of MCT is from about 0.1 g/kg to about 5 g/kg BW of the mammal. More preferably, the daily dose of MCT is from about 0.2 g/kg to about 3 g/kg of the mammal. Still more preferably, the daily dose of MCT is from about 0.5 g/kg to about 2 g/kg of the mammal.

### EXAMPLES

The following examples are provided for illustrative purposes only and are not intended to limit the scope of the invention.

### General Materials and Methods

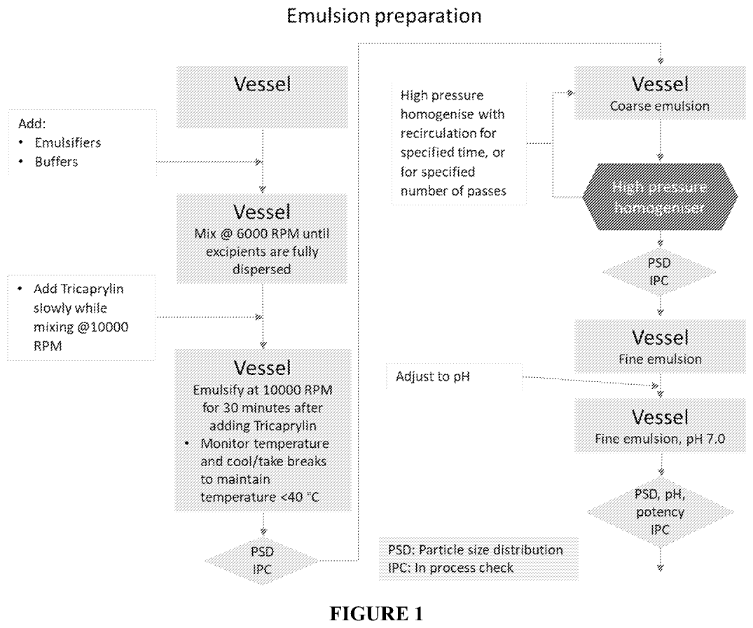

In accordance with the examples, exemplary liquid emulsions of the disclosure may be made according to the procedure illustrated in FIG. 1, with modifications as may be appropriate and understood by those of skill in the art.

In accordance with the examples, particle size testing methods that may be used are as follows:

### Procedure

1. Background measurement is performed with pure dispersant (milliQ-H₂O)
2. Vortex emulsion sample and add aliquots to dispersant with a pipette until the obscuration is within optimal range (5-15). The required volume is typically 10-100 μL depending on particle size with more volume required for smaller particle sizes. Wipe the outside of the pipette tip with a Kimwipe before addition to avoid any deposits forming on the outside of the tip due to evaporation.
3. Measurement is performed as 3 repeat sub-measurements and the result is reported as the average of 3 sub-measurements
4. Stirring rate of dispersion unit=3000 rpm
   Optical Properties of Materials (Selected from Database in Matersizer Software)
Lipid: Refractive Index 1.6; Absorption: 0.1
Dispersant (milliQ-H₂O): Refractive Index 1.33; Absorption: 0.1
   Equipment Used: Mastersizer 2000 with a Hydro 2000S dispersion unit; Malvern

### Measurement Times

Sample measurement time: 10 seconds
Background measurement time: 10 seconds
Sample measurement snaps: 10000
Background measurement snaps: 10000

### Measurement Cycles

Number of measurement cycles: 3
Delay between measurements: 10 seconds
Create average result from the 3 measurements

### Result Calculation Model

General purpose model
Calculation sensitivity: Normal (default)

In accordance with the examples, analytical testing methods that may be used are as follows:

| Test | Method | Draft Specifications |
|---|---|---|
| Description | Visual | White to off-white emulsion with no phase |
| | (in-house) | separation |
| Identification | (as per assay) | Retention time of main peak (C8) consistent with |
| | | reference standard |
| Assay | GC (in-house) | 90-110% of labelled claim |
| | HPLC (USP) | |
| Related Substances | (as per assay) | Report all peaks above 0.1% |
| Fatty Acid | GC (USP) | TBC |
| Composition | | |
| pH | USP <791>/Ph. Eur. | 6-7.5 |
| | 2.2.5 | |
| Particle Size | USP <429>/Ph.Eur. | Report results based on the method selected (Dv10, |
| Distribution | 2.9.31 | Dv50, Dv90 or MDD-mean droplet diameter) |
| Globule Size | USP <729> | |
| Distribution in Lipid | | |
| (Injectable) | | |
| Emulsions | | |
| Uniformity of | USP <905>/Ph.Eur. | Complies |
| Dosage Units | 2.9.40 | |
| Microbial Limits | USP <61>/Ph.Eur. | ≤1000 CFU/mL |
| Total Aerobic | 2.6.12 | ≤100 CFU/mL |
| Microbial Count | USP <61>/Ph.Eur. | Absent in 1 mL |
| (TAMC) | 2.6.12 | |
| Total Combined | USP <62>/Ph.Eur. | |
| Yeasts and Molds | 2.6.13 | |
| Count (TCYM) | | |
| Specific | | |
| Microorganisms: E. | | |
| coli | | |

| Test | Method |
|---|---|
| Assay and Related Substances | GC (in-house) |
| | HPLC (USP) |
| Fatty Acid Composition | GC (USP) |
| Particle Size Distribution | USP <429>/Ph.Eur. 2.9.31 |
| Globule Size Distribution in Lipid (Injectable) | USP <729> |
| Emulsions | |

### Example 1—Phospholipon 90G, Kolliphor RH40, Capmul PG-8 Emulsions

In various embodiments, liquid compositions of the disclosure may be formulated with combinations of emulsion forming agents such as Phospholipon 90G (Soy Lecithin), Kolliphor RH40 ((PEG-40 Hydrogenated Castor Oil), and Capmul PG-8 ((Propylene glycol monocaprylate).

Exemplary manufacturing methods for such formulations are generally as provided in FIG. 1 and are summarized in the table below:

| Step | Unit operation | CPP | In process check |
|---|---|---|---|
| 1 | Mix phospholipon 90G, kolliphor | Emulsifiers fully dispersed | Visual assessment of full dispersion |
| | RH40 in 50mM phosphate buffer | | |
| | pH 6.8 | | |
| 2 | Add tricaprylin oil slowly to the | Quantitative addition of oil | |
| | emulsifier-buffer mixture while | | |
| | high-shear mixing at 10.000 rpm | | |
| | with Silverson mixer | | |
| 3 | High shear mix for 30 minutes | Mixing speed | Measure temperature |
| | with Silverson mixer at 10.000 | Mixing time | Measure particle size distribution |
| | rpm | Batch size | after mixing |
| | | Temperature of solution | |
| | | during mixing (mixing | |
| | | process transfers heat to | Comment: 30-minute mixing time |
| | | solution) | found to be longer than required to |
| | | | achieve ultimate particle size |
| 4 | High pressure homogenise the | Number of passes | Measure particle size distribution |
| | emulsion for 10-12 passes at a | Homogenisation pressure | during process, e.g. after 7, 10, 12 |
| | target pressure of 7500 PSI | Discrete passes vs | passes |
| | | continuous recirculation | Comment: 7-12 passes found to be |
| | | | sufficient to achieve ultimate particle |
| | | | size |

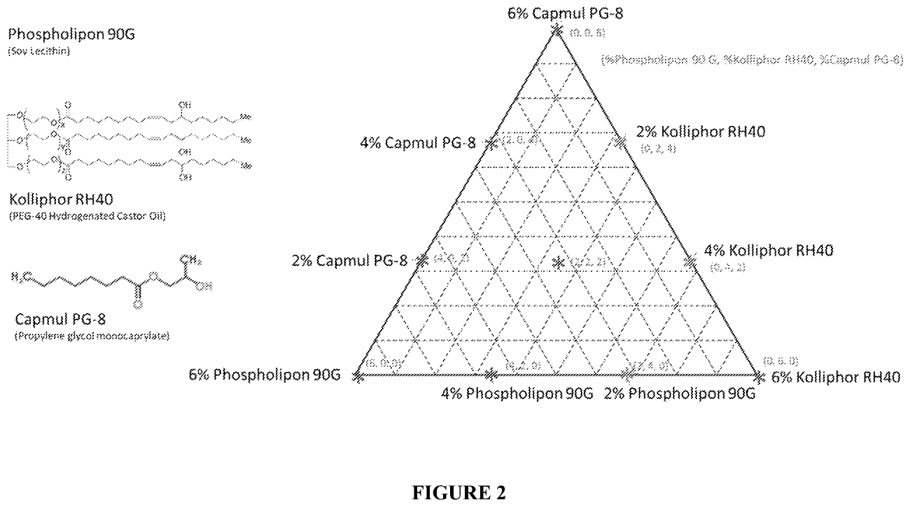

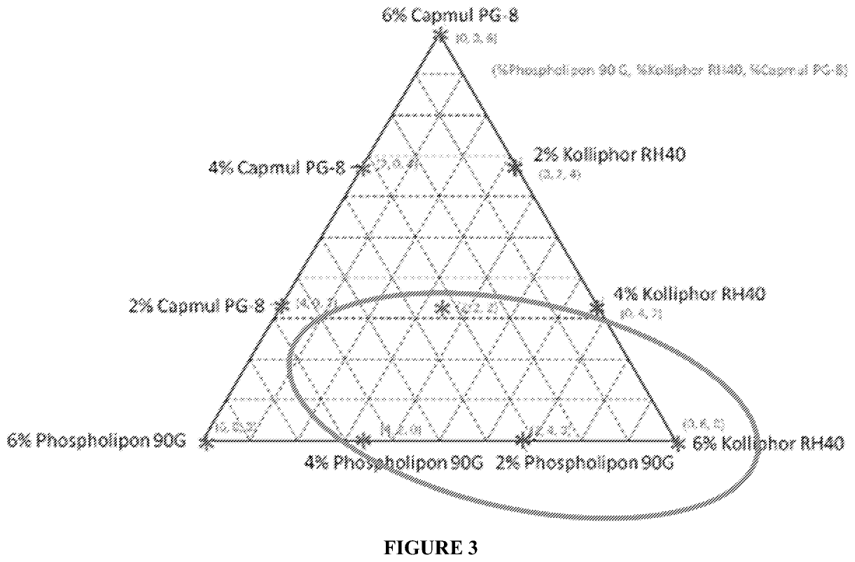

Exemplary emulsion forming agent concentrations are provided in FIG. 2, and stability results are shown in the table below and in FIG. 3.

Combinations of Phospholipon 90G, Kollipohor RH40 and Capmul PG-8

| | Composition | | | |
|---|---|---|---|---|
| MCT | Phospholipon | Kolliphor RH40 | Capmul PG-8 | |
| (%) | 90G (%) | (%) | (%) | Stability |
| 50 | 6 | 0 | 0 | + |
| 50 | 4 | 2 | 0 | +++ |
| 50 | 4 | 0 | 2 | − |
| 50 | 2 | 0 | 4 | − |
| 50 | 2 | 2 | 2 | +++ |
| 50 | 0 | 0 | 6 | − |
| 50 | 0 | 6 | 0 | ++ |

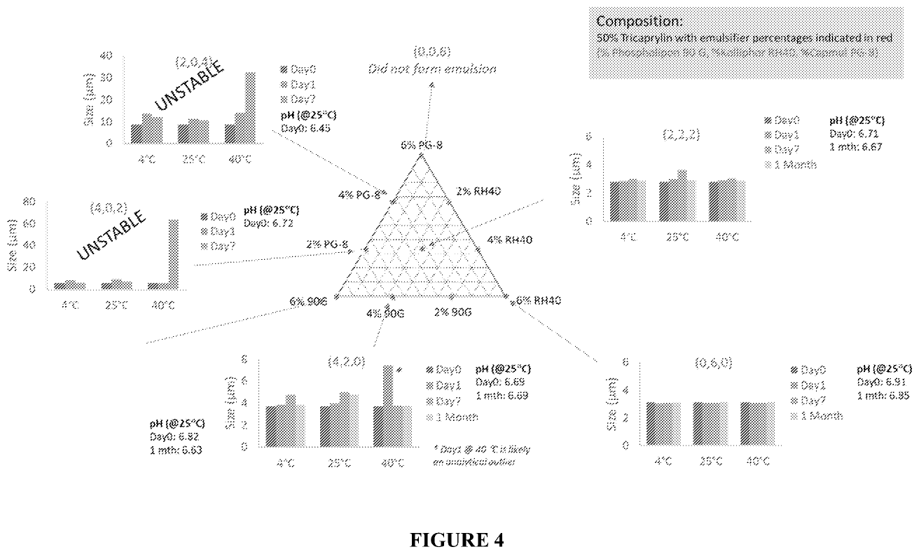

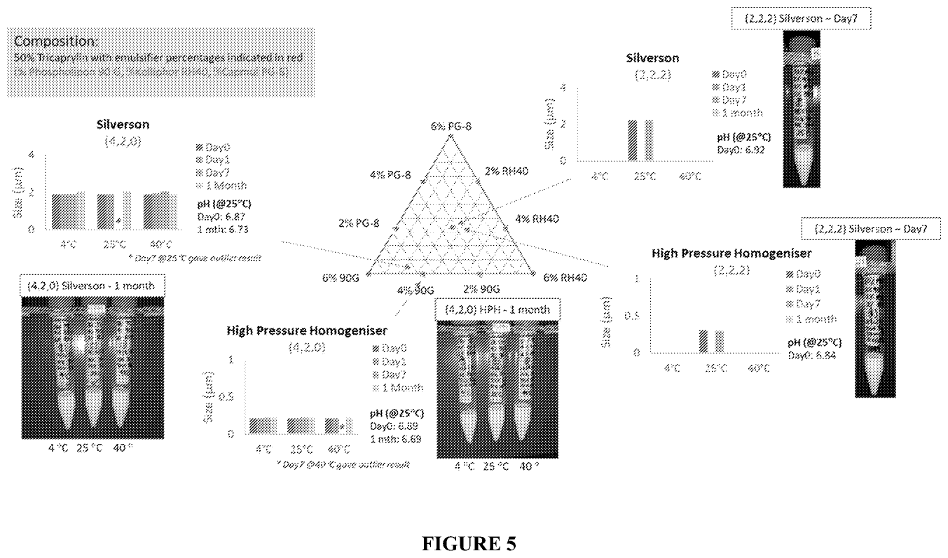

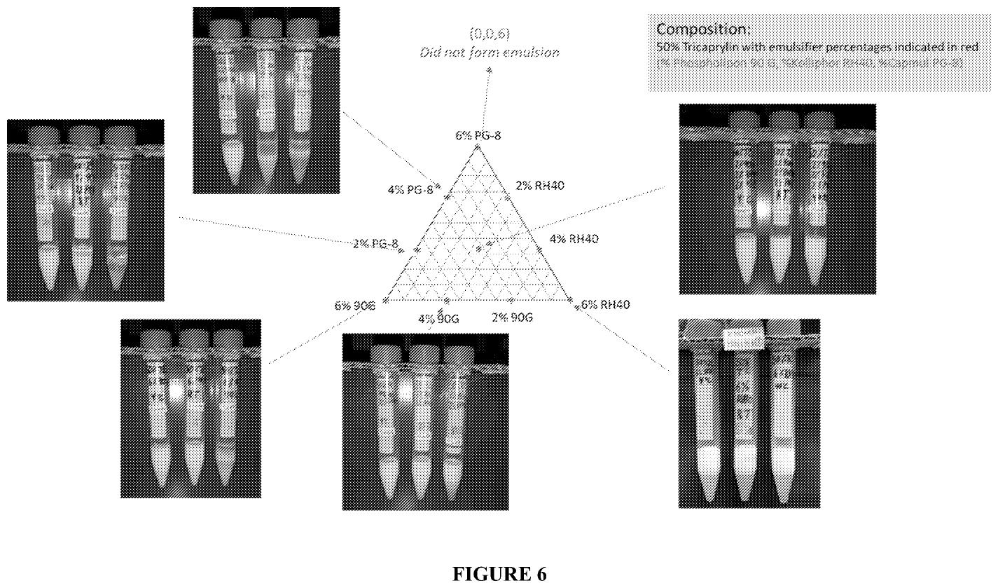

It was found that combinations and concentrations indicated in the circled portion of lower right-hand corner of FIG. 3 were found to be the most stable formulations. Equal mix of Phospholipon 90 G, Kolliphor RH40 and Capmul PG-8 was also very stable. Particle size evolution of Phospholipon 90G, Kolliphor RH40, Capmul PG-8 emulsions prepared via Ultra Turrax emulsification are shown in FIG. 4, and particle size evolution of 10 Phospholipon 90G, Kolliphor RH40, Capmul PG-8 emulsions: prepared via Silverson & High Pressure Homogeniser emulsification are shown in FIG. 5. Visual appearance of Phospholipon 90G, Kolliphor RH40, Capmul PG-8 emulsions under a 1 month stability study are shown in FIG. 6.

Chemical stability of exemplary formulations are shown below:

| | | Caprylic Acid | |
|---|---|---|---|
| | | (mg/ml) | |
| | | Day 0 | Day 7 |
| Formulation | Processing | RT | 25° C. |
| 50% MCT, 4% 90G, 2% RH40 | Silverson + High Pressure Homogeniser | 0 | 0 |
| 50% TC, 2.67% 90G, 1.33% RH40 | Silverson + High Pressure Homogeniser | 0 | 0 |
| 50% MCT, 6% RH40 | Silverson + High Pressure Homogeniser | 0 | 0 |
| 50% TC, 2% 90G, 2% RH40, 2% PGS | Silverson + High Pressure Homogeniser | 1.500 | 1.600 |
| | | (v0.3% of TC) | (v0.32% of TC) |

Caprylic acid was not detected in the 50% Tricaprylin 2% Phospholipon 90G, 2% Kolliphor RH40, 2% Capmul PG-8 examples. Caprylic acid was not detected in any of the examples without Capmul PG-8.

A summary of findings is provided below:

| | | Initial | | |
|---|---|---|---|---|
| | | particle | | |
| Formulation | Processing | size | Physical Stability | Chemical Stability |
| 50% Tricaprylin, | Silverson | ≈1.9 μm | Particle size | N/A |
| 4% Phospholipon 90 G, | | | virtually unchanged | |
| | | | for 1 month at all | |
| | | | storage conditions. | |
| | | | No creaming. | |
| 2% Kolliphor RH40 | High | ≈0.22 μm | Particle size | Caprylic acid not detected at |
| | pressure | | virtually unchanged | 0 days and 7 day storage at |
| | homogeniser | | for 1 month at all | 25 C. |
| | | | storage conditions. | |
| | | | No creaming. | |
| 50% Tricaprylin, | Silverson | ≈2.4 μm | Particle size ≈ 2.5 | N/A |
| 2.67% Phospholipon | | | μm after 7 days at | |
| 90G, 1.33% Kolliphor | | | 25° C. | |
| RH40 | High | ≈0.29 μm | Particle size ≈ 0.31 | Not yet tested |
| (Total emulsifier | pressure | | μm after 7 days at | |
| concentration 4%) | homogeniser | | 25° C. | |
| 50% Tricaprylin, | Silverson | ≈2.2 μm | Particle size ≈ 2.2 | |
| 2% Phospholipon 90 G, | | | μm after 7 days at | |
| | | | 25° C. | |
| 2% Kolliphor RH40, | High | ≈0.31 μm | Particle size ≈ 0.29 | Caprylic acid detected at 1.6 |
| 2% Capmul PG-8 | pressure | | μm after 7 days at | mg/mL after 7 days at 25° C. |
| | homogeniser | | 25° C. | (≈0.3% of tricaprylin |
| | | | | concentration) |

Overall, both 50% Tricaprylin, 4% Phospholipon 90 G, 2% Kolliphor RH40 and 50% Tricaprylin, 2% Phospholipon 90 G, 2% Kolliphor RH40, 2% Capmul PG-8 have very good physical stability at one month. Caprylic acid was detected in 50% Tricaprylin, 2% Phospholipon 90 G, 2% Kolliphor RH40, 2% Capmul PG-8 sample at one month. Reducing total emulsifier concentration to 4% in the 50% Tricaprylin, 2.67% Phospholipon 90G, 1.33% Kolliphor RH40 only had a minor effect on initial particle size.

In other examples, the impact of Kolliphor RH40 concentrations was investigated. Kolliphor content was reduced by increasing Lecithin-kolliphor ratio and/or decreasing total emulsifier content Results are shown below:

| | Composition | | | | | | | |
|---|---|---|---|---|---|---|---|---|
| | Phospholipon | Kolliphor | | Particle size (μm) | | | | |
| MCT | 90G | RH40 | Glycerol | | Day 7 | | 1 Month | |
| (%) | (%) | (%) | (%) | Day 0 | 25° C. | 40° C. | 25° C. | 40° C. |
| 50 | 5.5 | 0.5 | 0 | 0.385 | 0.390 | 8.03 | 1.563 | 41.011 |
| 50 | 5 | 1 | 0 | 0.321 | 0.326 | 43.476 | 0.326 | 23.239 |
| 50 | 5 | 1 | 2.5 | 0.290 | 0.465 | 25.727 | 0.341 | 6.42 |
| | | | | | (D9) | (D9) | | |
| 50 | 4 | 2 | 0 | 0.217 | 0.221 | — | 0.223 | 0.22 |
| 50 | 4 | 2 | 2.5 | 0.200 | 0.209 | 0.331 | 0.205 | 0.204 |
| | | | | | (D9) | (D9) | | |
| 50 | 2.67 | 1.33 | 0 | 0.294 | 0.307 | | 0.306* | |
| | | | | | | | (6 | |
| | | | | | | | weeks) | |
| 20 | 1.6 | 0.8 | 0 | 0.180 | 0.182 | 0.181 | 0.182 | 0.182 |
| 20 | 1.6 | 0.8 | 2.5 | 0.182 | 0.183 | 0.183 | 0.185 | 0.185 |

### Example 2—Citrem and Monoglycerides and Diglycerides of Fatty Acids Emulsions

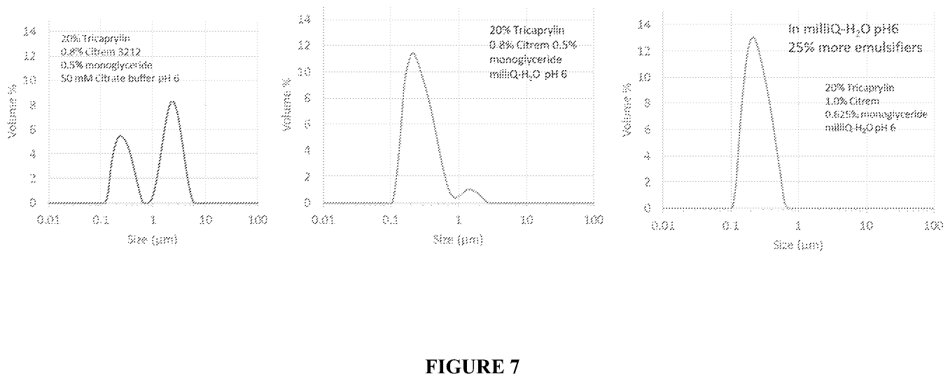

In various embodiments, liquid compositions of the disclosure may be formulated with combinations of emulsion forming agents such as Citrem and monoglycerides and diglycerides of fatty acids. Emulsions prepared include: 20% Tricaprylin, 0.8% Citrem, 0.5% monoglyceride in Citrate Buffer pH 6 (2 formulations made using emulsifiers from different suppliers); 20% Tricaprylin, 0.8% Citrem, 0.5% monoglyceride in milliQ-H20 pH 6; and 20% Tricaprylin, 0.8% Citrem, 0.5% monoglyceride in milliQ-H20 pH 6. Exemplary particle size distributions are shown in FIG. 7.

### Example 3—Additional Component Combinations

Additional investigations were performed to improve stability and MCT concentration. Results are shown below:

### Stabilizing Strategies

| Strategy | Formulation | Stability | Progress further |
|---|---|---|---|
| Include CMC | 20% Tricaprylin, 2.4% | Creaming instability | No |
| to Increase viscosity | 90G, 2.5% Glycerol, 0.5% | | |
| | CMC | | |
| Include Safflower oil | 20% Tricaprylin. 2.4% | No clear improvement | No |
| to reduce Ostwald | 90G, 2.5% Glycerol, 1% | | |
| | Safflower oil | | |
| ripening | 20% Tricaprylin, 2.4% | No clear improvement | No |
| | 90G, 2.5% Glycerol, 5% | | |
| | Safflower oil | | |
| Increase surface | 20% Tricaprylin, 2.4% | Unstable | No |
| charge by raising pH | 90G, 2.5% Glycerol, | | |
| | 0.06% Sodium Oleate pH& | | |
| of oleate containing | 20% Tricaprylin, 2.4% | Stable at 25° C. | Yes-in combination with |
| emulsions | 90G, 2.5% Glycerol, 0.5% | Unstable at 40° C. | other emulsifiers |
| | Sodium Oleate pH8 | | |
| Include citrem to | 20% Tricaprylin, 1.6% | Good short term | Yes |
| increase surface | 90G, 0.8% Citrem, 2.5% | stability at 25° C. and | |
| charge | Glycerol | 40° C. | |

### Increase Tricaprylin and Optimize Stability

| Strategy | Formulation | Stability |
|---|---|---|
| Combination Kolliphor | 20% Tricaprylin, 2.4% 90G, 0.2% | Stable at 25° C. |
| RH40 and oleate at pH8 | Kolliphor RH40 2.5% Glycerol | Unstable at 40° C. |
| Increase tricaprylin content | 50% Tricaprylin, 4% 90G, 2% | Sample very viscous, unlikely to be a |
| | Citrem, 2.5% Glycerol | viable product |
| of stable citrem formulation | 40% Tricaprylin, 3.2% 90G, 1.6% | |
| | Citrem, 2.5% Glycerol | |

Additional Formulations have been investigated, including 40% Tricaprylin, 3.2% 90G, 1.6% Citrem, 2.5% Glycerol; 40% Tricaprylin, 2.13% 90G, 1.07% Citrem, 2.5% Glycerol; 40% Tricaprylin, 2.13% 90G, 1.07% RH40; 40% Tricaprylin, 2.13% 90G, 1.07% RH40, 2.5% Glycerol; 40% Tricaprylin, 4.8% 90G, 2.5% Glycerol, 1% Sodium oleate, 0.4% RH40 pH8; 50% TC, 4% 90G, 2% RH40; 40% Tricaprylin, 2.13% 90G, 1.07% RH40; 40% Tricaprylin, 2.13% 90G, 1.07% RH40, 2.5% Glycerol; 20% TC, 2.4% 90G, 2.5% Glycerol, 0.5% Sodium oleate, 0.2% RH40; 40% TC, 3.2% 90G, 1.6% Citrem, 2.5% Glycerol; and 40% TC, 3.2% 90G, 1.6% RH40.

### Comparative Example 4: Lecithin, Sodium Oleate, Glycerol Emulsifiers

In other embodiments, exemplary liquid formulations of the disclosure may be prepared from combinations of emulsion forming agents such as: Lecithin, sodium oleate and glycerol ratios at different MCT concentrations. The formulations were prepared by: Silverson vs High pressure homogenization.

| | | | % Emulsifier | | | | | |
|---|---|---|---|---|---|---|---|---|
| | | | 2.4% Lecithin | | | 1.6% Lecithin | | |
| | | | 0% | 0.06% | 0.12% | 0% | 0.06% | 0.12% |
| | | | Oleate | Oleate | Oleate | Oleate | Oleate | Oleate |
| % Tricaprylin | 20% | 0% | X | X | — | X | — | — |
| | | Glycerol | | | | | | |
| | | 2.5% | X | X | X | — | — | — |
| | | Glycerol | | | | | | |
| | | | 6% Lecithin | | | 4% Lecithin | | |
| | | | 0% | 0.15% | 0.3% | 0% | 0.15% | 0.3% |
| | | | Oleate | Oleate | Oleate | Oleate | Oleate | Oleate |
| | 50% | 0% | X | X | — | x | — | — |
| | | Glycerol | | | | | | |
| | | 2.5% | X | X | X | — | — | x |
| | | Glycerol | | | | | | |

(—) Formulations planned but not prepared based on unsatisfactory results of other formulations unlikely to meet 12-month stability at 25° C.

Emulsions reasonably stable but unlikely to meet requirement of 12-month stability at 25° C. Sodium oleate had a slight destabilizing effect at pH7. Overall, these formulations were not pursued further based on stability results.

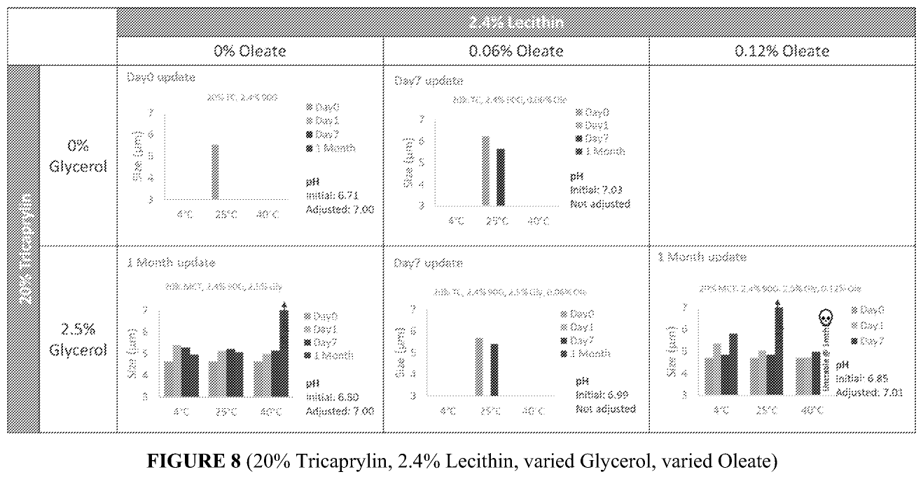

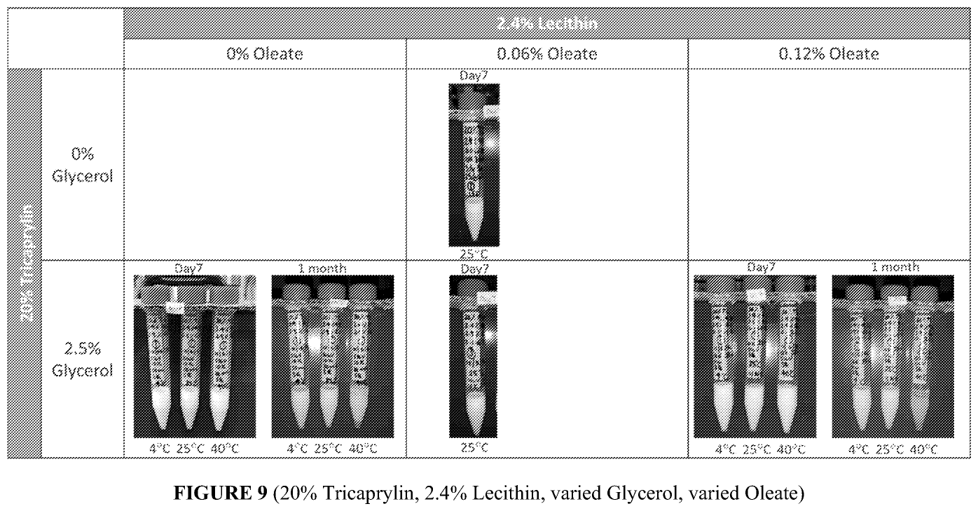

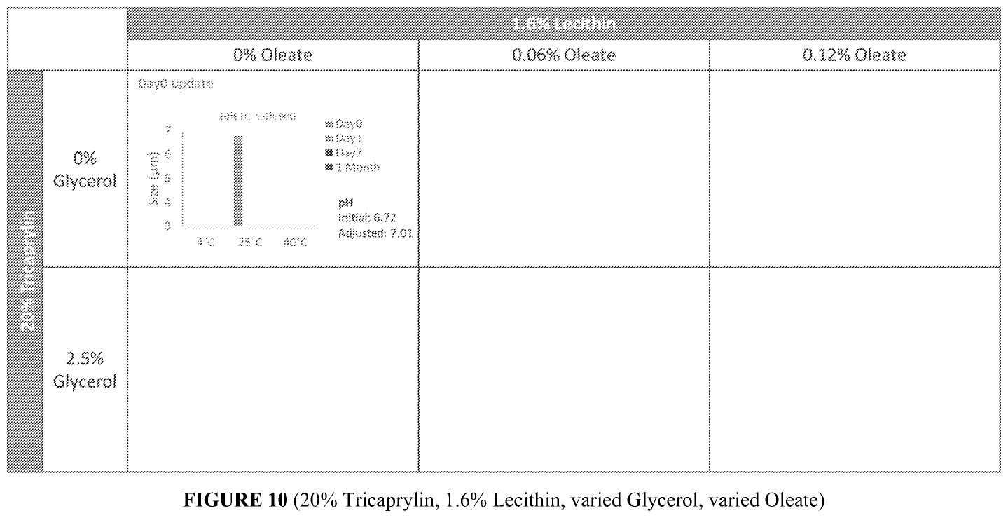

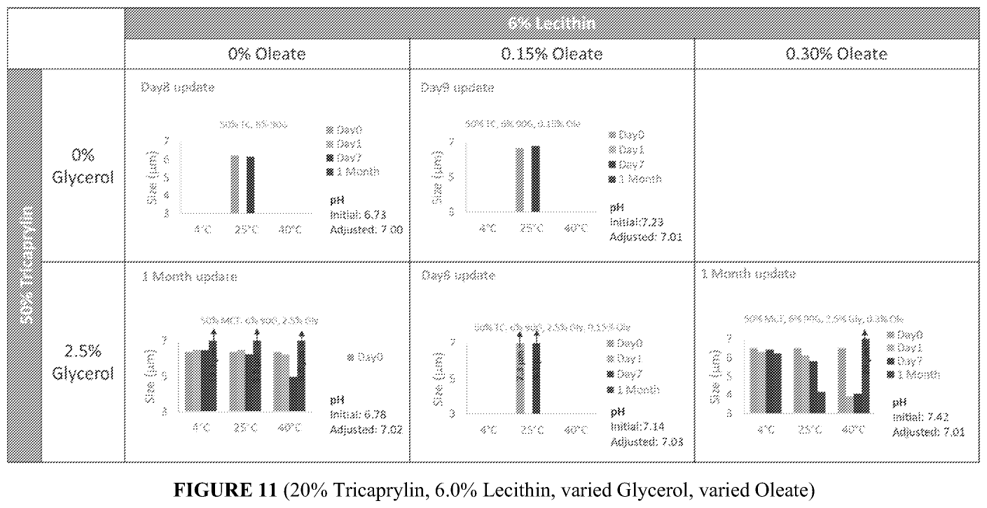

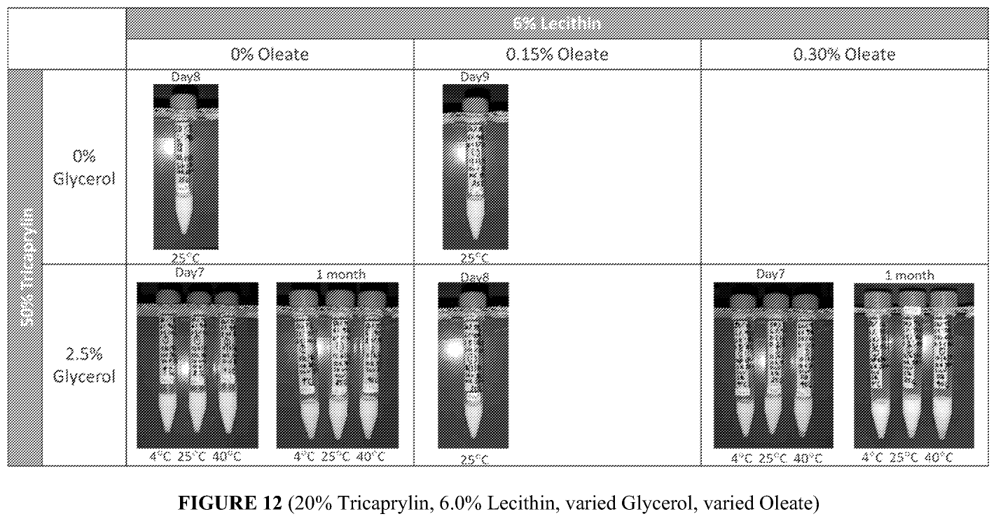

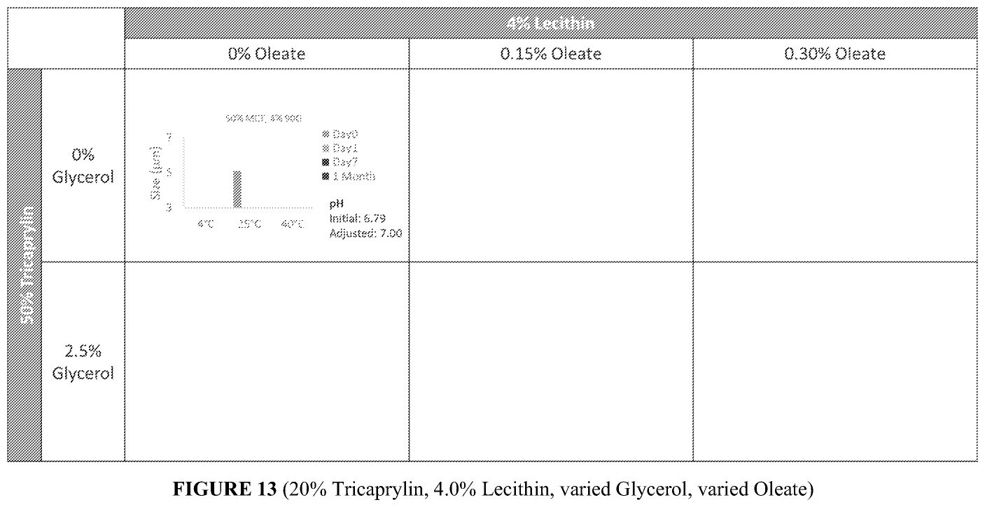

Stability results for formulations prepared via a Silverson mixer are shown in FIG. 8 (Stability results for 20% Tricaprylin, 2.4% Lecithin, varied Glycerol, varied Oleate), FIG. 9 (Visual stability results for 20% Tricaprylin, 2.4% Lecithin, varied Glycerol, varied Oleate), FIG. 10 (Stability results for 20% Tricaprylin, 1.6% Lecithin, varied Glycerol, varied Oleate), FIG. 11 (Stability results for 20% Tricaprylin, 6.0% Lecithin, varied Glycerol, varied Oleate, FIG. 12 (Visual stability results for 20% Tricaprylin, 6.0% Lecithin, varied Glycerol, varied Oleate, and FIG. 13 (Stability results for 20% Tricaprylin, 4.0% Lecithin, varied Glycerol, varied Oleate).

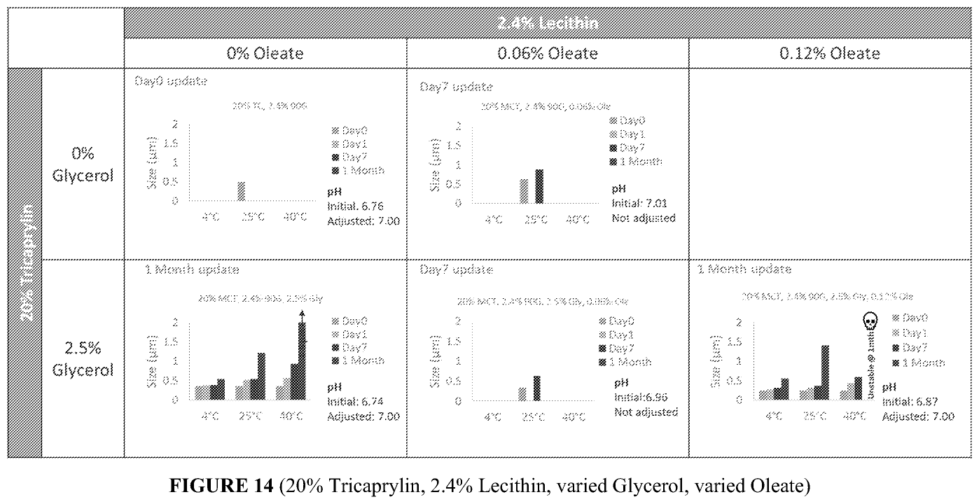

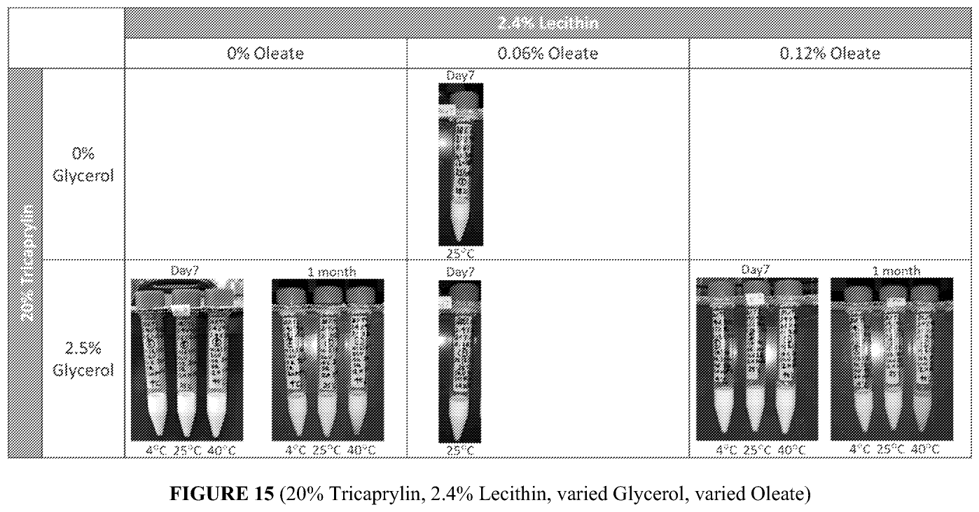

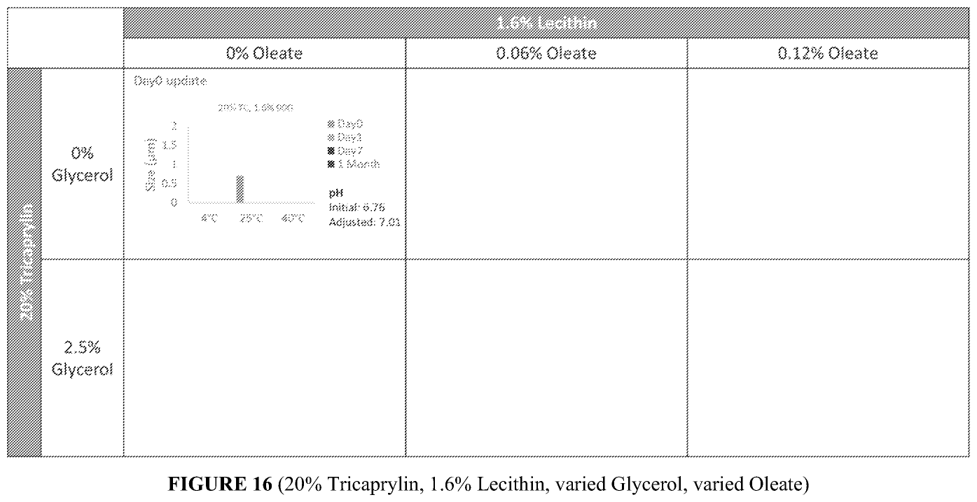

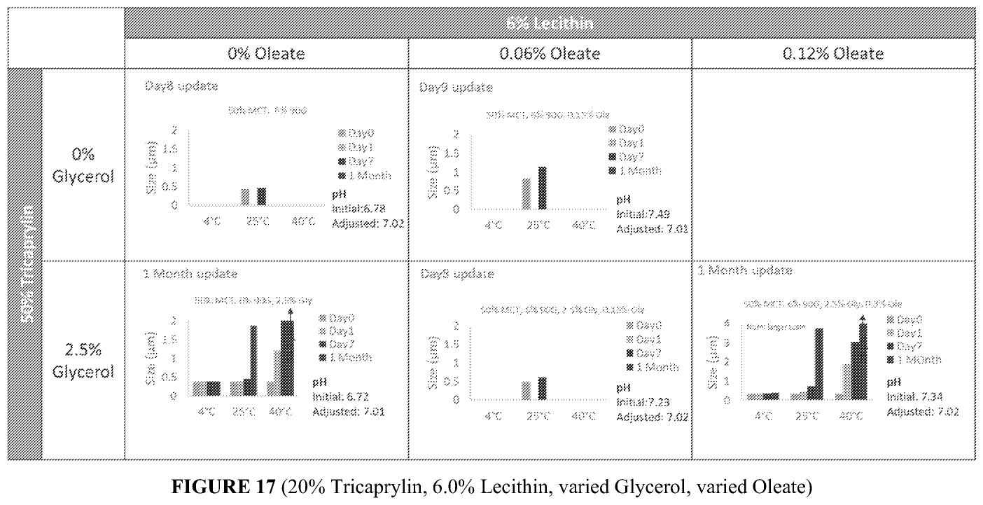

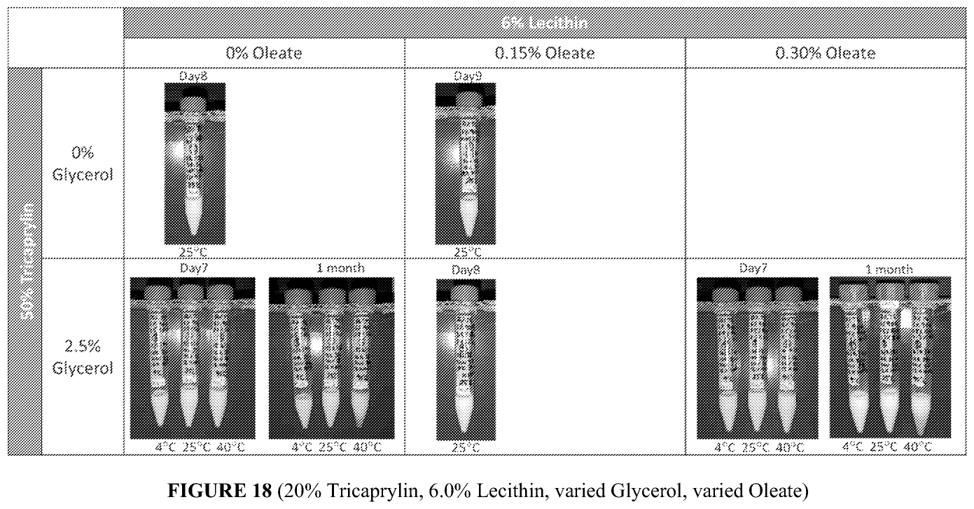

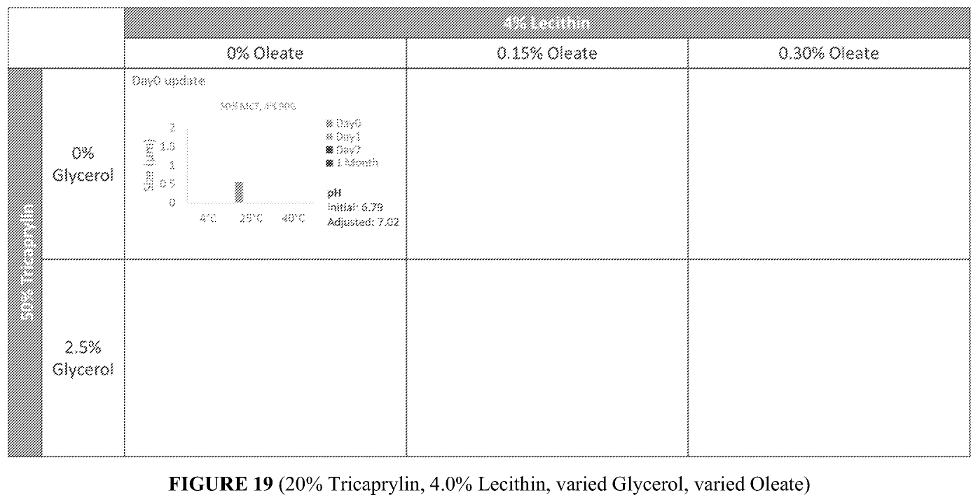

Stability results for formulations prepared via a High Pressure homogenation are shown in FIG. 14 (Stability results for 20% Tricaprylin, 2.4% Lecithin, varied Glycerol, varied Oleate), FIG. 15 (Visual stability results for 20% Tricaprylin, 2.4% Lecithin, varied Glycerol, varied Oleate), FIG. 16 (Stability results for 20% Tricaprylin, 1.6% Lecithin, varied Glycerol, varied Oleate), FIG. 17 (Stability results for 20% Tricaprylin, 6.0% Lecithin, varied Glycerol, varied Oleate, FIG. 18 (Visual stability results for 20% Tricaprylin, 6.0% Lecithin, varied Glycerol, varied Oleate, and FIG. 19 (Stability results for 20% Tricaprylin, 4.0% Lecithin, varied Glycerol, varied Oleate).

### Example 5—Bioburden Reduction Methods

Methods for bioburden reduction of the formulations of the disclosure are provided below:

Method

1. Gamma Irradiation
3. Autoclave or Dry Heat Oven
5. Ultra Heat Treatment-like
6. Filtration

As part of the development process, a series of bioburden reduction measures were evaluated for their effectiveness and potential to be incorporated into the manufacturing process. Details of the various studies performed are summarized below.

### Filtration

Incorporation of filtration as a bioburden reduction step may be used as the simplest method of reducing bio load (if present). Filter compatibility studies were conducted by Pall Filtration, with evaluation of microbial retention of the selected filters conducted. These studies confirmed that selected formulations were sensitive to the type of filter media, and that the preferred media type was not microbially retentive.

### Dry Heat

Heating is a commonly accepted method of reducing bio load. Evaluation of heat conditions commonly used to control microbial growth was attempted on exemplary formulations. All temperature conditions trialed showed significant disruption to the PSD profile of the product, confirming that the application of heat is not viable for bioburden control. This observation correlates with the deterioration of product due to elevated temperature observed in the development trials.

### Gamma Irradiation

Gamma irradiation is a commonly accepted method of sterilizing componentry and reducing bio load in natural materials. Exemplary formulations were irradiated at various dosages and then assessed for the impact of irradiation on product performance. This study confirmed that a 10 kG dosage was effective in achieving a 6-log reduction in bio load, with no impact to the product critical quality attributes tested.

The following table depict the impact of various doses of gamma irradiation on Tricaprylin as well as a representative formulation (“formulation 2” 50% Tricaprylin, 2.67% Phospholipon 90G, 1.33% Kolliphor RH 40, 50 mM Phosphate buffer pH 6.8).

| | | | Test & Result /Description | | |
|---|---|---|---|---|---|
| | Physiochemical | | After Treatment | | |
| | and bioburden | | Note failure in Appearance, | | |
| | testing | | (discoloration or phase split), | | |
| Sample & | Pre & post | Dosage | Particle Size Distribution, Assay or | | |
| Description | treatment | (kGy) | Related Substances. | | Complies/fails |
| Tricaprylin | Note: | 10 | Appearance: | No change from | Complies |
| | Visual: (discoloration, | | | pre-irradiated | |
| | Phase split) | | | sample | |
| | Particle size | | Particle size | NA | NA |
| | distribution | | distribution: | | |
| | Assay & Related | | Assay: | No change from | Complies |
| | substances profile. | | | pre-irradiated | |
| | TAMC, TYMC: | | | sample | |
| | NB: Viscosity & | | Related | Related | Complies* |
| | Escherichia coli not | | substances: | Substance | |
| | tested due to | | | profile changes | |
| | insufficient sample | | pH: NA- non | NA | NA |
| | availability. | | aqueous | | |
| | Tricaprilin API is a | | TAMC: | No growth | Complies |
| | non-aqueous oil, so | | | observed | |
| | pH test not relevant | | TYMC: | No growth | Complies |
| | | | | observed | |
| | | 15 | Appearance: | No change from | Complies |
| | | | | pre-irradiated | |
| | | | | sample | |
| | | | Particle size | NA | NA |
| | | | distribution: | | |
| | | | Assay: | No change from | Complies |
| | | | | pre-irradiated | |
| | | | | sample | |
| | | | Related | Related | Complies* |
| | | | substances: | Substance | |
| | | | | profile changes | |
| | | | pH: NA- non | NA | NA |
| | | | aqueous | | |
| | | | TAMC: | No growth | Complies |
| | | | | observed | |
| | | | TYMC: | No growth | Complies |
| | | | | observed | |
| | | 25 | Appearance: | No change from | Complies |
| | | | | pre-irradiated | |
| | | | | sample | |
| | | | Particle size | NA | NA |
| | | | distribution: | | |
| | | | Assay: | No change from | Complies |
| | | | | pre-irradiated | |
| | | | | sample | |
| | | | Related | Related | Complies* |
| | | | substances: | Substance | |
| | | | | profile changes | |
| | | | pH: NA- non | NA | NA |
| | | | aqueous | | |
| | | | TAMC: | No growth | Complies |
| | | | | observed | |
| | | | TYMC: | No growth | Complies |
| | | | | observed | |
| Formulation 2 | Note: | 10 | Appearance: | No change from | Complies |
| | Visual: (discoloration, | | | pre-irradiated | |
| | Phase split) | | | sample | |
| | Particle size | | Particle size | No change from | Complies |
| | distribution | | distribution: | pre-irradiated | |
| | Assay & Related | | | sample | |
| | substances profile. | | Assay: | No change from | Complies |
| | pH, viscosity | | | pre-irradiated | |
| | TAMC, TYMC: | | | sample | |
| | Escherichia coli | | Related | No change from | Complies |
| | NB: Viscosity & | | substances: | pre-irradiated | |
| | Escherichia coli not | | | sample | |
| | tested due to | | pH: | No change from | Complies |
| | insufficient sample | | | pre-irradiated | |
| | availability | | | sample | |
| | | | TAMC: | No growth | Complies |
| | | | | observed | |
| | | | TYMC: | No growth | Complies |
| | | | | observed | |
| | | 15 | Appearance: | No change from | Complies |
| | | | | pre-irradiated | |
| | | | | sample | |
| | | | Particle size | PSD profile | Fails |
| | | | distribution: | changed | |
| | | | Assay: | No change from | Complies |
| | | | | pre-irradiated | |
| | | | | sample | |
| | | | Related | No change from | Complies |
| | | | substances: | pre-irradiated | |
| | | | | sample | |
| | | | pH: | No change from | Complies |
| | | | | pre-irradiated | |
| | | | | sample | |
| | | | TAMC: | No growth | Complies |
| | | | | observed | |
| | | | TYMC: | No growth | Complies |
| | | | | observed | |
| | | 25 | Appearance: | No change from | Complies |
| | | | | pre-irradiated | |
| | | | | sample | |
| | | | Particle size | PSD profile | Fails |
| | | | distribution: | changed | |
| | | | Assay: | No change from | Complies |
| | | | | pre-irradiated | |
| | | | | sample | |
| | | | Related | minor change | Complies** |
| | | | substances: | from pre- | |
| | | | | irradiated | |
| | | | | sample | |
| | | | pH: | No change from | Complies |
| | | | | pre-irradiated | |
| | | | | sample | |
| | | | TAMC: | No growth | Complies |
| | | | | observed | |
| | | | TYMC: | No growth | Complies |
| | | | | observed | |
| Biological | G. stearothermophilus | 10 | NA | No Growth | Complies |
| indicator | indicator strips | 15 | | No Growth | Complies |
| | Strip spore loading | 25 | | No Growth | Complies |
| | NLT 1 × 10⁶ | | | | |

No microbes were detected in Formulation 2 prior or post gamma irradiation. The results obtained from the biological indicator strips confirmed that gamma irradiation was successful as a 6-log reduction of bioburden was achieved at all doses tested.

Physio-chemical evaluation of formulation samples irradiated at 10 kGy confirmed no change in appearance, assay, related substances profile, particle size distribution or pH. Irradiated samples also showed no negative impact from gamma irradiation on the primary container and closures for all doses evaluated, i.e., seals were not observed to become brittle as a result of irradiation

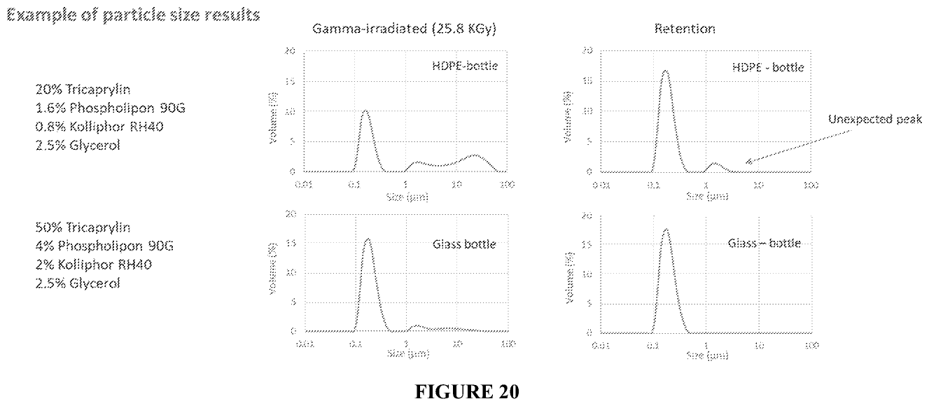

Samples irradiated at 15 and 25 kGy also showed no changes to appearance, assay and pH, however the related substance profile and particle size distribution were impacted at these doses. In this regard, particle size distribution of gamma-irradiated samples appear more affected than retention samples, results shown in FIG. 20.

### Example 6—Exemplary and Comparative Formulations

The following formulations of the disclosure and comparative formulations were prepared in accordance with the general methodology of FIG. 1, as may be modified as required and understood by those of skill in the art.

| | |
|---|---|
| Formulation | Comments |

Phospholipon 90G-Kolliphor RH40-Capmul PG-8 emulsions

| | |
|---|---|
| 50% Tricaprylin, 6% 90G | Acceptable |
| 50% Tricaprylin, 4% 90G, 2% RH40 | Good stability |
| 50% Tricaprylin, 4% 90G, 2% RH40, | Good stability |
| 2.5% Glycerol | |
| 50% Tricaprylin, 2% 90G, 4% RH40 | Acceptable |
| 50% Tricaprylin, 6% Kolliphor RH40 | Good stability |
| 50% Tricaprylin, 4% 90G, 2% Capmul | Unstable |
| PG-8 | |
| 50% Tricaprylin, 2% 90G, 4% Capmul | Unstable |
| PG-8 | |
| 50% Tricaprylin, 6% Capmul PG-8 | Did not form emulsion |
| 50% Tricaprylin, 4% Kolliphor RH40, | Capmul did not generally improve emulsion stability |
| 2% Capmul PG-8 | |
| 50% Tricaprylin, 2% Kolliphor RH40, | Capmul did not generally improve emulsion stability |
| 4% Capmul PG-8 | |
| 50% Tricaprylin, 2% 90G, 2% Kolliphor | Good stability. |
| RH40, 2% Capmul PG-8 | |
| 50% Tricaprylin, 5.5% 90G, 0.5% RH40 | Acceptable |
| 50% Tricaprylin, 5% 90G, 1% RH40 | Acceptable |
| 50% Tricaprylin, 5% 90G, 1% RH40, | Acceptable |
| 2.5% Glycerol | |
| 50% Tricaprylin, 2.67% 90G, 1.33% | Good stability |
| RH40 | |
| 20% Tricaprylin, 1.6% 90G, 0.8% RH40 | Good stability |
| 20% Tricaprylin, 1.6% 90G, 0.8% RH40, | Good stability |
| 2.5% Glycerol | |
| 50% Tricaprylin , 4% 90G, 2% RH40 | Emulsified at 50° C. to see effect of emulsification |
| | temperature. Effect was insignificant. |
| 50% Tricaprylin, 2% 90G, 2% RH40, 2% | Emulsified at 50° C. to see effect of emulsification |
| Capmul PG-8 | temperature. Effect was insignificant. |

| | |
|---|---|
| Formulation | Comments |

Citrem-monoglyceride emulsions

| | |
|---|---|
| 20% Tricaprylin, 0.8% Citrem (3212), | Poor initial particle size distribution |
| 0.5% monoglyceride (DMG 0291), 50 | |
| mM citrate buffer pH6 | |
| 20% Tricaprylin, 0.8% Citrem (3212), | Fair initial particle size distribution |
| 0.5% monoglyceride (DMG 0291), | |
| milliQ-H2O pH6 | |
| 20% Tricaprylin, 1.0% Citrem (3212), | Good initial particle size distribution |
| 0.625% monoglyceride (DMG 0291), | |
| milliQ-H2O pH6 | |
| 20% Tricaprylin, 0.8% Citrem (KV- | Fair initial particle size distribution |
| 30E), 0.5% monoglyceride (P(V)S), 50 | |
| mM citrate buffer pH6 | |

| | |
|---|---|
| Formulation | Comments |

Formulations with varying compositions aiming to elucidate major instability mechanisms
and to combine factors that appear favourable to emulsion stability

| | |
|---|---|
| 20% Tricaprylin, 2.4% Phospholipon | CMC added with the aim of increasing viscosity and |
| 90G, 2.5% Glycerol, 0.5% | |
| Carboxymethylcellulose pH 7 | slow down gravitational separation. Phase separated |
| | after 12 days, possibly due to bridging flocculation. |
| 20% Tricaprylin, 2.4% Phospholipon | Safflower oil added as an oil with very low water |
| 90G, 2.5% Glycerol, 1% Safflower oil | solubility with the aim of inhibiting Ostwald |
| pH 7 | ripening. |
| 20% Tricaprylin, 2.4% Phospholipon | Safflower oil added as an oil with very low water |
| 90G, 2.5% Glycerol, 5% Safflower oil | |
| pH 7 | solubility with the aim of inhibiting Ostwald |
| | ripening. 5-fold higher amount than above to see |
| | effect of concentration. |
| 20% Tricaprylin, 2.4% Phospholipon | pH raised to pH8 should increase the charge of |
| 90G, 0.06% Sodium Oleate, 2.5% | sodium oleate and potentially improve stability due |
| Glycerol, pH 8 | to electrostatic forces. Poor stability. |
| 20% Tricaprylin, 2.4% Phospholipon | pH raised to pH8 should increase the charge of |
| 90G, 0.5% Sodium Oleate, 2.5% | sodium oleate and potentially improve stability due |
| Glycerol, pH 8 | to electrostatic forces. Prepared with larger sodium |
| | oleate amount than above to see effect of sodium |
| | oleate content. Poor stability. |
| 20% Tricaprylin, 2.4% Phospholipon | Excellent short term stability at 25° C. and very small |
| 90G, 0.2% Kolliphor RH40, 0.5% | particle size with narrow particle size distribution. |
| Sodium Oleate, 2.5% Glycerol, pH 8 | Short term stability data indicates lower stability at |
| | 40° C. |
| 20% Tricaprylin, 1.6% Phospholipon | Good initial particle size distribution. |
| 90G, 0.8% Citrem (3212), 2.5% | |
| Glycerol, pH 7 | |

| | |
|---|---|
| Formulation | Comments |

Phospholipon 90G-Sodium Oleate-Glycerol emulsions

| | |
|---|---|
| 50% Tricaprylin, 6% 90G pH7 | Reasonably stable |
| 50% Tricaprylin, 6% 90G, 2.5% | Reasonably stable |
| Glycerol pH7 | |
| 50% Tricaprylin, 6% 90G, 0.15% Oleate | Reasonably stable |
| pH7 | |
| 50% Tricaprylin, 6% 90G, 0.15% Oleate, | Reasonably stable |
| 2.5% Glycerol pH7 | |
| 50% Tricaprylin, 6% 90G, 0. 30% Oleate | Sodium oleate did not improve |
| pH7 | stability at pH 7 |
| 50% Tricaprylin, 6% 90G, 0.30% Oleate, | Reasonably stable |
| 2.5% Glycerol pH7 | |
| 20% Tricaprylin, 2.4% 90G pH7 | Reasonably stable |
| 20% Tricaprylin, 2.4% 90G, 2.5% | Reasonably stable |
| Glycerol pH7 | |
| 20% Tricaprylin, 2.4% 90G, 0.06% | Reasonably stable |
| Oleate pH7 | |
| 20% Tricaprylin, 2.4% 90G, 0.06% | Reasonably stable |
| Oleate, 2.5% Glycerol pH7 | |
| 20% Tricaprylin, 2.4% 90G, 0. 12% | Sodium oleate did not improve |
| Oleate pH7 | stability at pH 7 |
| 20% Tricaprylin, 2.4% 90G, 0.06% | Reasonably stable |
| Oleate, 2.5% Glycerol pH7 | |
| 10% Tricaprylin, 1.2% 90G, 2.5% | |
| Glycerol, 0.03% a-Tocopherol, 0.03% | |
| Oleate | |
| 20% Tricaprylin, 2.4% 90G, 2.5% | |
| Glycerol, 0.06% a-Tocopherol, 0.06% | |
| Oleate | |

### Example 7-Longer Term Stability

The following formulations of the disclosure and comparative formulations were prepared in accordance with the general methodology of FIG. 1, as may be modified as required and understood by those of skill in the art. Exemplary longer term stability results are illustrated in the below tables.

Lecithin: Kolliphor RH40 - Particle size

| | Particle size (μm) | | | | | | | | |
|---|---|---|---|---|---|---|---|---|---|
| | | Day 7 | | 1 Month | | 3 months | | 6 months | |
| Formulation | Day 0 | 25° C. | 40° C. | 25° C. | 40° C. | 25° C. | 40° C. | 25° C. | 40° C. |
| 50% Tricaprylin, 5.5% 90G, 0.5% RH40 | 0.385 | 0.390 | 8.03 | 1.563 | 41.011 | 0.530 | 27.188 | 12.655 | Unstable |
| 50% Tricaprylin, 5% 90G, 1% RH40 | 0.321 | 0.326 | 43.48 | 0.326 | 23.239 | 0.319 | 7.097 | 0.431 | Unstable |
| 50% Tricaprylin, 5% 90G, 1% RH40, 2.5% Glycerol | 0.290 | 0.465 [symbol] | 25.73 [symbol] | 0.341 | 28.965 | 61.196 | 10.016 | 29.851 | Unstable |
| 50% Tricaprylin, 4% 90G, 2% RH40 [AC-OLE-02] | 0.217 | 0.221 | NM | 0.223 | 0.220 | 0.225 | 0.649 [symbol] | 0.227 | NM |
| 50% Tricaprylin, 4% 90G, 2% RH40 [AC-OLE-02] | 0.188 | NM | NM | 0.189 | 0.188 | 0.203 | 0.203 | 0.281 | 0.427 |
| 50% Tricaprylin, 4% 90G, 2% RH40, | 0.200 | 0.209 [symbol] | 0.331 [symbol] | 0.205 | 0.204 | 0.206 | 0.302 | 0.207 | NM |
| 2.5% Glycerol [AC-OLE-01] | | | | | | | | | |
| 50% Tricaprylin, 2.67% 90G, 1.33% RH40 | 0.294 | 0.307 | NM | 0.306 [symbol] | 1.786 [symbol] | 0.556 | NM | 0.654 | NM |
| 40% Tricaprylin, 3.2% 90G, 1.6% RH40 [AC-OLE-04] | 0.178 | 0.179 | 0.178 | 0.178 | 0.179 | 0.179 | 0.178 | 0.183 | 0.548 |
| 40% Tricaprylin, 3.2% 90G, 1.6% RH40, | 0.179 | 0.180 [symbol] | 0.192 [symbol] | 0.181 | 0.180 | 0.180 | 0.218 | 0.186 | 0.702 |
| 2.5% Glycerol [AC-OLE-09] | | | | | | | | | |
| 40% Tricaprylin, 2.13% 90G, 1.07% RH40 [AC-OLE-05] | 0.230 | 0.238 | 0.237 | 0.251 | 0.237 | 0.254 | 0.968 | 0.276 | 0.683 |
| 40% Tricaprylin, 2.13% 90G, 1.07% RH40, | 0.231 | 0.235 | 0.232 | 0.236 | 0.298 | 0.238 | 2.161 | 0.246 | 0.733 |
| 2.5% Glycerol [AC-OLE-03] | | | | | | | | | |
| 20% Tricaprylin, 1.6% 90G, 0.8% RH40 [AC-OLE-10] | 0.180 | 0.182 | 0.181 | 0.182 | 0.182 | 0.183 | 0.233 | 0.183 | NM |
| 20% Tricaprylin, 1.6% 90G, 0.8% RH40, | 0.182 | 0.183 | 0.183 | 0.185 | 0.185 | 0.192 | 0.254 | 0.186 | NM |
| 2.5% Glycerol [AC-OLE-07] | | | | | | | | | |

[symbol]

indicates data missing or illegible when filed

Lecithin: Kolliphor RH40 -pH

| | pH | | | | | | | | |
|---|---|---|---|---|---|---|---|---|---|
| | | Day 7 | | 1 Month | | 3 months | | 6 months | |
| Formulation | Day 0 | 25° C. | 40° C. | 25° C. | 40° C. | 25° C. | 40° C. | 25° C. | 40° C. |
| 50% Tricaprylin, 5.5% 90G, 0.5% RH40 | 6.8 | 6.8 | 6.8 | 5.7 | 6.7 | 7.2 | 4.1 | 5.5 | Unstable |
| 50% Tricaprylin, 5% 90G, 1% RH40 | 6.8 | 6.8 | 6.8 | 6.8 | 6.4 | 7.0 | 3.8 | 6.9 | Unstable |
| 50% Tricaprylin, 5% 90G, 1% RH40, 2.5% Glycerol | 6.8 | 6.7 | 6.9 | 5.2 | 6.4 | 5.6 | 3.8 | 4.3 | Unstable |
| 50% Tricaprylin, 4% 90G, 2% RH40 [AC-OLE-02] | 6.9 | 6.6 | 6.6 | 6.7 | 6.7 | 6.9 | 6.3 | 6.9 | NM |
| 50% Tricaprylin, 4% 90G, 2% RH40 [AC-OLE-02] | 7.0 | NM | NM | 6.8 | 7.0 | 6.8 | 6.8 | 6.6 | 4.0 |
| 50% Tricaprylin, 4% 90G, 2% RH40, | 6.8 | 6.9 | 7.0 | 6.8 | 6.8 | 6.9 | 4.1 | 6.9 | NM |
| 2.5% Glycerol [AC-OLE-01] | | | | | | | | | |
| 50% Tricaprylin, 2.67% 90G, 1.33% RH40 | 6.9 | 6.7 | NM | 6.9 | NM | 6.9 | NM | 6.9 | NM |
| 40% Tricaprylin, 3.2% 90G, 1.6% RH40 [AC-OLE-04] | 6.9 | 6.9 | 6.9 | 6.9 | 6.8 | 6.9 | 6.7 | 6.8 | 4.0 |
| 40% Tricaprylin, 3.2% 90G, 1.6% RH40, | 6.8 | 6.9 | 6.9 | 6.8 | 6.9 | 6.8 | 4.9 | 6.6 | 3.7 |
| 2.5% Glycerol [AC-OLE-09] | | | | | | | | | |
| 40% Tricaprylin, 2.13% 90G, 1.07% RH40 \|AC-OLE-05] | 6.8 | 6.9 | 6.9 | 6.9 | 6.9 | 6.8 | 6.7 | 6.7 | 4.3 |
| 40% Tricaprylin, 2.13% 90G, 1.07% RH40, | 6.8 | 6.9 | 6.9 | 7.0 | 7.0 | 6.8 | 6.1 | 6.6 | 4.3 |
| 2.5% Glycerol [AC-OLE-03] | | | | | | | | | |
| 20% Tricaprylin, 1.6% 90G, 0.8% RH40 [AC-OLE-10] | 6.8 | 6.8 | 6.8 | 6.9 | 6.9 | 7.1 | 6.1 | 6.9 | NM |
| 20% Tricaprylin, 1.6% 90G, 0.8% RH40, | 6.8 | 6.8 | 6.8 | 6.8 | 6.9 | 7.2 | 6.0 | 6.9 | NM |
| 2.5% Glycerol [AC-OLE-07] | | | | | | | | | |

Lecithin: Kolliphor RH40 - Chemical Stability

| | Caprylic Acid (mg/mL) | | | | | | |
|---|---|---|---|---|---|---|---|
| | | Day 7 | | 1 Month | | 3 months | |
| Formulation | Day 0 | 25° C. | 40° C. | 25° C. | 40° C. | 25° C. | 40° C. |
| 50% Tricaprylin, 5.5% 90G, 0.5% RH40 | 0 | 0 | 0 | <LOQ | 0 | 0 | <LOQ |
| 50% Tricaprylin, 5% 90G, 1% RH40 | 0 | 0 | 0 | 0 | 0 | 0 | 1.1 |
| 50% Tricaprylin, 5% 90G, 1% RH40, 2.5% Glycerol | 0 | 0 [symbol] | 0 [symbol] | <LOQ | 0 | 5.7 | 0.57 |
| 50% Tricaprylin, 4% 90G, 2% RH40 [AC-OLE-02] | 0 | 0 | NM | 0 | 0 | 0 | <LOD |
| 50% Tricaprylin, 4% 90G, 2% RH40 [AC-OLE-02] | 0 | NM | NM | <LOD | 0 | 0.83 | 0 |
| 50% Tricaprylin, 4% 90G, 2% RH40, 2.5% Glycerol | 0 | 0 [symbol] | 0 [symbol] | 0 | 0 | 0 | 0 |
| 50% Tricaprylin, 2.67% 90G, 1.33% RH40 | 0 | 0 | NM | 0 [symbol] | NM | 0 | NM |
| 40% Tricaprylin, 3.2% 90G, 1.6% RH40 [AC-OLE-04] | 0 | NM | NM | 0 | 0 | 0 | 0 |
| 40% Tricaprylin, 3.2% 90G, 1.6% RH40, | 0 | NM | NM | 0 | 0 | 0 | <LOQ |
| 2.5% Glycerol [AC-OLE-09] | | | | | | | |
| 40% Tricaprylin, 2.13% 90G, 1.07% RH40 [AC-OLE-05] | 0 | NM | NM | 0 | 0 | 0 | 0 |
| 40% Tricaprylin, 2.13% 90G, 1.07% RH40, | 0 | NM | NM | 0 | 0 | 0 | 0 |
| 2.5% Glycerol [AC-OLE-03] | | | | | | | |
| 20% Tricaprylin, 1.6% 90G, 0.8% RH40 [AC-OLE-10] | 0 | 0 | 0 | 0 | 0 | 0 | 0 |
| 20% Tricaprylin, 1.6% 90G, 0.8% RH40, | 0 | 0 | 0 | 0 | 0 | 0 | <LOQ |
| 2.5% Glycerol [AC-OLE-07] | | | | | | | |

| | Caprylic Acid (mg/mL) | | | | |
|---|---|---|---|---|---|
| | 6 months | | 12 months | | |
| Formulation | 25° C. | 40° C. | 4° C. | 25° C. | 40° C. |
| 50% Tricaprylin, 5.5% 90G, 0.5% RH40 | <LOQ | NM | Unstable | Unstable | Unstable |
| 50% Tricaprylin, 5% 90G, 1% RH40 | 0 | NM | NM | Unstable | NM |
| 50% Tricaprylin, 5% 90G, 1% RH40, 2.5% Glycerol | <LOQ | NM | 0 | 0.41 | NM |
| 50% Tricaprylin, 4% 90G, 2% RH40 [AC-OLE-02] | 0 | NM | NM | <LOQ | NM |
| 50% Tricaprylin, 4% 90G, 2% RH40 [AC-OLE-02] | 1.7 | 0.39 | | | |
| 50% Tricaprylin, 4% 90G, 2% RH40, 2.5% Glycerol | 0 | NM | NM | NM | [symbol] |
| 50% Tricaprylin, 2.67% 90G, 1.33% RH40 | 0 | NM | NM | 0 | [symbol] |
| 40% Tricaprylin, 3.2% 90G, 1.6% RH40 [AC-OLE-04] | <LOD | 0.86 | 0 | 0.35 | [symbol] |
| 40% Tricaprylin, 3.2% 90G, 1.6% RH40, | 0 | 1.68 | | | [symbol] |
| 2.5% Glycerol [AC-OLE-09] | | | | | |
| 40% Tricaprylin, 2.13% 90G, 1.07% RH40 [AC-OLE-05] | <LOD | 0.63 | | | [symbol] |
| 40% Tricaprylin, 2.13% 90G, 1.07% RH40, | <LOO | 0.68 | | | [symbol] |
| 2.5% Glycerol [AC-OLE-03] | | | | | |
| 20% Tricaprylin, 1.6% 90G, 0.8% RH40 [AC-OLE-10] | 0 | NM | 0 | 0 | [symbol] |
| 20% Tricaprylin, 1.6% 90G, 0.8% RH40, | 0 | NM | 0 | 0 | [symbol] |
| 2.5% Glycerol [AC-OLE-07] | | | | | |

[symbol]

indicates data missing or illegible when filed

Lecithin: Kolliphor RH40, Oleate, pH8 - Low Kolliphor Content: Particle Size and pH

| | | Day 7 | | 1 Month | | 3 Months | | 6 Months | |
|---|---|---|---|---|---|---|---|---|---|
| Formulation | Day 0 | 25° C. | 40° C. | 25° C. | 40° C. | 25° C. | 40° C. | 25° C. | 40° C. |
| | Particle size (μm) | | | | | | | | |
| 40% Tricaprylin, 4.8% 90G, 0.4% RH40, | 0.183 | 0.206 | 23.434 | 0.258* | 21.587* | 2.051 | 25.853 | 1.948 | 25.771 |
| 1% Oleate, 2.5% Glycerol, pH8 | | | | (Day22) | (Day22) | | | | |
| 20% Tricaprylin, 2.4% 90G, | 0.177 | 0.179 | 0.497 | 0.181 | 0.491 | 0.184 | 0.652 | 0.210 | Unstable |
| 0.2% RH40, 0.5% Oleate, | | | | | | | | | |
| 2.5% Glycerol, pH 8 [AC-OLE-08] | | | | | | | | | |
| 40% Tricaprylin, 4.8% 90G, 1% | 0.208 | 0.431 | 38.417 | 0.634 [symbol] | Unstable [symbol] | Unstable | Unstable | Unstable | Unstable |
| Oleate, 2.5% Glycerol, pH8 | | | | (Day24) | (Day24) | | | | |
| 20% Tricaprylin, 2.4% 90G, 0.5% | 0.226 | 0.465 [symbol] | 13.675 | 0.249 | 11.332 | 0.461 | 8.749 | 0.742 | Unstable |
| Oleate, 2.5% Glycerol, pH8 | pH | | | | | | | | |
| 40% Tricaprylin, 4.8% 90G, 0.4% RH40, | 8.0 | 8.0 | 7.7 | 8.0* | 7.5* | 7.4 | 6.0 | 6.6 | 5.1 |
| 1% Oleate, 2.5% Glycerol, pH8 | | | | (Day22) | (Day22) | | | | |
| 20% Tricaprylin [symbol] 2.4% | 8.0 | 8.0 | 7.7 | 7.8 | 6.5 | 7.5 | 6.4 | 6.8 | Unstable |
| 90G, 0.2% RH40, 0.5% Oleate, | | | | | | | | | |
| 2.5% Glycerol, pH 8 [AC-OLE-08] | | | | | | | | | |
| 40% Tricaprylin, 4.8% 90G, 1% | 8.0 | 8.1 | 8.0 | 8.2 [symbol] | Unstable [symbol] | 7.5 | 6.2 | 7.0 | 5.7 |
| Oleate, 2.5% Glycerol, pH8 | | | | (Day24) | (Day24) | | | | |
| 20% Tricaprylin, 2.4% 90G, 0.5% | 8.1 | 7.7 | 7.2 | 7.8 | 7.2 | 7.3 | 6.4 | 6.5 | Unstable |
| Oleate, 2.5% Glycerol, pH8 | | | | | | | | | |

[symbol]

indicates data missing or illegible when filed

Lecithin: Kolliphor RH40, Oleate, pH8 - Low Kolliphor Content: Chemical Stability

| | Caprylic Acid (mg/mL) | | | | | | | | |
|---|---|---|---|---|---|---|---|---|---|
| | | Day 7 | | 1 Month | | 3 months | | 6 months | |
| Formulation | Day 0 | 25° C. | 40° C. | 25° C. | 40° C. | 25° C. | 40° C. | 25° C. | 40° C. |
| 40% Tricaprylin, 4.8% 90G, 0.4% RH40, 1% Oleate, 2.5% | 0 | NM | NM | 0* | <LOD* | 1.00 | 2.04 | <LOD | 5.13 |
| Glycerol, pH8 | | | | (Day22) | (Day22) | | | | |
| 20% Tricaprylin, 2.4% 90G, 0.2% RH40, 0.5% Oleate, 2.5% | 0 | 0 | 0 | 0 | <LOD | <LOD | 0.92 | 0.17 | Unstable |
| Glycerol, pH 8 | | | | | | | | | |
| 40% Tricaprylin, 4.8% 90G, 1% Oleate, 2.5% Glycerol, pH8 | 0 | NM | NM | 0 [symbol] | <LOD [symbol] | <LOD | 1.79 | <LOQ | 4.15 |
| | | | | (Day24) | (Day24) | | | | |
| 20% Tricaprylin, 2.4% 90G, 0.5% Oleate, 2.5% Glycerol, | NM | 0 | 0 | 0 | <LOD | <LOQ | 0.85 | 0.12 | NM |
| pH8 | | | | | | | | | |

[symbol]

indicates data missing or illegible when filed

Lecithin:Citrem - Particle Size and pH

| | | Day 7 | | 1 Month | | 3 Months | | 6 Months | | 12 Months | | |
|---|---|---|---|---|---|---|---|---|---|---|---|---|
| Formulation | Day 0 | 25° C. | 40° C. | 25° C. | 40° C. | 25° C. | 40° C. | 25° C. | 40° C. | 4° C. | 25° C. | 40° C. |
| | Particle size (μm) | | | | | | | | | | | |
| 40% Tricaprylin, 3.2% | 0.247 | 0.287 | 0.485 | 0.337 | 0.528 | 0.502 | Unstable | 10.004 | Unstable | | | |
| 90G, 1.6% Citrem, 2.5% | | | | | | | | | | | | |
| Glycerol, pH 7 [AC-OLE-06] | | | | | | | | | | | | |
| 40% Tricaprylin, 2.13% | 0.329 | NM | NM | 0.358* | 0.571* | 0.720 | 1.212 | 0.752 | Unstable | | | |
| 90G, 1.07% Citrem, | | | | | | | | | | | | |
| 2.5% Glycerol, pH 7 | | | | (Day20) | (Day20) | | | | | | | |
| 20% Tricaprylin, 1.6% | 0.226 | 0.231 | 0.255 | 0.237 | 0.268 | 0.239 | 0.305 | 0.259 | 0.444 | 0.260 | 0.398 | Unstable |
| 90G, 0.8% Citrem, | | | | | | | | | | | | |
| 2.5% Glycerol, pH 7 | | | | | | | | | | | | |
| | pH (μm) | | | | | | | | | | | |
| 40% Tricaprylin, 3.2% | 7.0 | 7.1 | 7.0 | 7.0 | 6.9 | 6.7 | 6.1 | 6.2 | 5.7 | | | |
| 90G, 1.6% Citrem, 2.5% | | | | | | | | | | | | |
| Glycerol, pH 7 [AC-OLE-06] | | | | | | | | | | | | |
| 40% Tricaprylin [symbol] | 7.0 | NM | NM | 6.9* | 7.0* | 6.7 | 6.5 | 6.3 | 6.1 | | | |
| 2.13% 90G, 1.07% Citrem, | | | | | | | | | | | | |
| 2.5% Glycerol, pH 7 | | | | (Day20) | (Day20) | | | | | | | |
| 20% Tricaprylin, 1.6% | 7.0 | 7.1 | 7.0 | 7.0 | 6.8 | 7.1 | 6.9 | 6.9 | 6.6 | 6.9 | 6.9 | Unstable |
| 90G, 0.8% Citrem, 2.5% | | | | | | | | | | | | |
| Glycerol, pH 7 | | | | | | | | | | | | |

[symbol]

indicates data missing or illegible when filed

Lecithin:Citrem - Chemical Stability

| | Caprylic Acid (mg/mL) | | | | | | | | | | | |
|---|---|---|---|---|---|---|---|---|---|---|---|---|
| | | Day 7 | | 1 Month | | 3 months | | 6 months | | 12 months | | |
| Formulation | Day 0 | 25° C. | 40° C. | 25° C. | 40° C. | 25° C. | 40° C. | 25° C. | 40° C. | 4° C. | 25° C. | 40° C. |
| 40% Tricaprylin, 3.2% 90G, 1.6% | 0 | NM | NM | 0.51 | <LOD | 1.83 | 4.43 | 5.14 | 3.73 | | | |
| Citrem, 2.5% Glycerol, pH 7 | | | | | | | | | | | | |
| 40% Tricaprylin, 2.13% 90G, 1.07% | 0 | NM | NM | 1.15 | 0.41 | 2.22 | 0.88 | 3.69 | 2.13 | | | |
| Citrem, 2.5% Glycerol, pH 7 | | | | | | | | | | | | |
| 20% Tricaprylin, 1.6% 90G, 0.8% | 0 | NM | NM | 0 | 0 | 0 | 0 | 0 | <LOQ | 0 | 0 | NM |
| Citrem, 2.5% Glycerol, pH 7 | | | | | | | | | | | | |

### Example 8—Flavouring

Additional investigations were performed to improve taste and overall palatability of the liquid pharmaceutical formulations of the disclosure. Various sweetening and flavouring agents were investigated. Results are shown below:

### Sweetening Agent

Three levels of sucralose (0.025%, 0.05% and 0.1%) and three levels of stevia (0.1%, 0.2% and 0.3%) were compared. No clear sweetening difference between sucralose and stevia was identified, as the flavouring agent predominates. However, sucralose is slightly preferred as it can be used at lower concentrations (i.e., 0.05%-0.1% sucralose was found to provide a sufficient level of sweetness).

### Flavouring Agent

Different flavouring agents were compared (at a 0.1% sucralose concentration). Concentrations were selected based on manufacturers recommendations and initial trials.

| Supplier | Supplier ID | Solubility | Concentration | Observations |
|---|---|---|---|---|
| Givaudan Vanilla 1 | L-132184 | Water | 0.15% | −− |
| Sensient Vanilla 1 | AA000244 | Water | 0.3% | + |
| Sensient Vanilla 2 | QAA0016060002 | Oil | 0.2% | ++ slightly milky |
| Scentral Vanilla 1 | FL0131 | Water | 0.2% | + slightly strong |
| Scentral Vanilla 2 | FL0141 | Oil | 0.2% | ++ also slightly strong |

It was unexpectedly found that oil soluble flavouring agents providing improved taste and overall palatability to the liquid pharmaceutical formulations of the disclosure.

### Example 9—Lead Formulation Selection

Based on the above formulation and comparative formulation preparation, the following lead formulations were selected:

| | | Concentration | |
|---|---|---|---|
| No | Formulation | of Tricaprylin | Excipients Used |
| AC-OLE-1 | 50% Tricaprylin, 4% 90G, | 50% | Phospholipon 90G, Koliphor |
| | 2% RH40, 2.5% Gly | | RH40, Glycerol |
| AC-OLE-2 | 50% Tricaprylin, 4% 90G, | 50% | Phospholipon 90G, Koliphor |
| | 2% RH40 | | RH40 |
| AC-OLE-3 | 40% Tricaprylin, 2.13% | 40% | Phospholipon 90G, Koliphor |
| | 90G, 1.07% RH40, 2.5% | | RH40, Glycerol |
| | Gly | | |
| AC-OLE-4 | 40% Tricaprylin, 3.2% | 40% | Phospholipon 90G, Koliphor |
| | 90G, 1.6% RH40 | | RH40 |
| AC-OLE-5 | 40% Tricaprylin, 2.13% | 40% | Phospholipon 90G, Koliphor |
| | 90G, 1.07% RH40 | | RH40 |
| AC-OLE-6 | 40% Tricaprylin, 3.2% | 40% | Phospholipon 90G, Citrem |
| | 90G, 1.6% Citrem 3212, | | 3122, Glycerol |
| | 2.5% Gly | | |
| AC-OLE-7 | 20% Tricaprylin, 1.6% | 20% | Phospholipon 90G, Koliphor |
| | 90G, 0.8% RH40, 2.5% | | RH40 |
| | Glycerol | | |
| AC-OLE-8 | 20% Tricaprylin. 2.4% | 20% | Phospholipon 90G, Koliphor |
| | 90G, 2.5% Gly, 0.5% | | RH40, Sodium Oleate, Glycerol |
| | Oleate, 0.2% RH40 | | |
| AC-OLE-9 | 40% Tricaprylin, 3.2% | 40% | Phospholipon 90G, Koliphor |
| | 90G, 1.6% RH40, 2.5% | | RH40, Glycerol |
| | Gly | | |
| AC-OLE-10 | 20% Tricaprylin, 1.6% | 20% | Phospholipon 90G, Koliphor |
| | 90G, 0.8% RH40 | | RH40 |

Lead formulation selection rationale:
Physical and chemical stability primary factor
Range of Tricaprilin concentrations and presence and absence of glycerol, as well as a combination of excipients selected as these factors could potentially affect PK profile
All lead formulations display relatively fast digestion profile.
Citrem formulations, while being physically and chemically stable may pose some manufacturing related issues (particle size could be affected by filtration, GC analysis).
Sodium oleate formulations have been shown to be temperature sensitive, and could pose some manufacturing and storage related constrains. Nonetheless the formulations have still chosen as selected lead candidates as these represent a combination of different excipients which could provide interesting variations from a PK and tolerability standpoint.

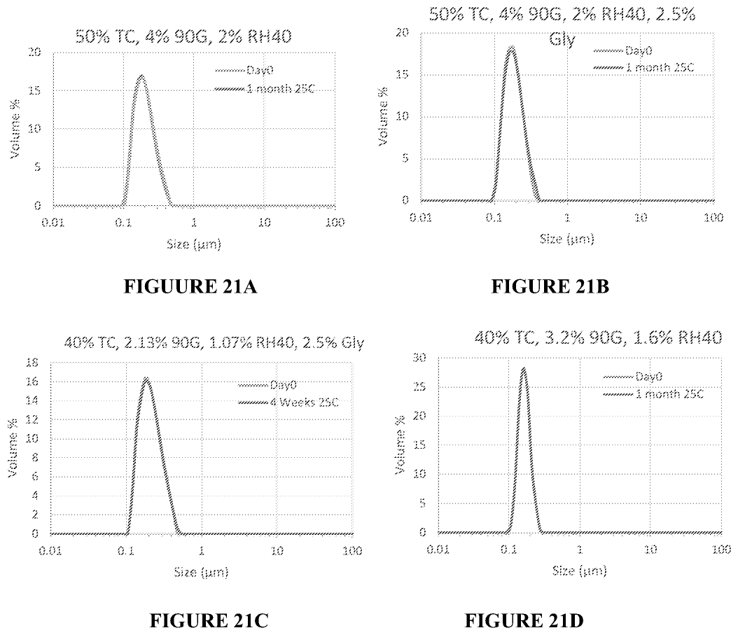

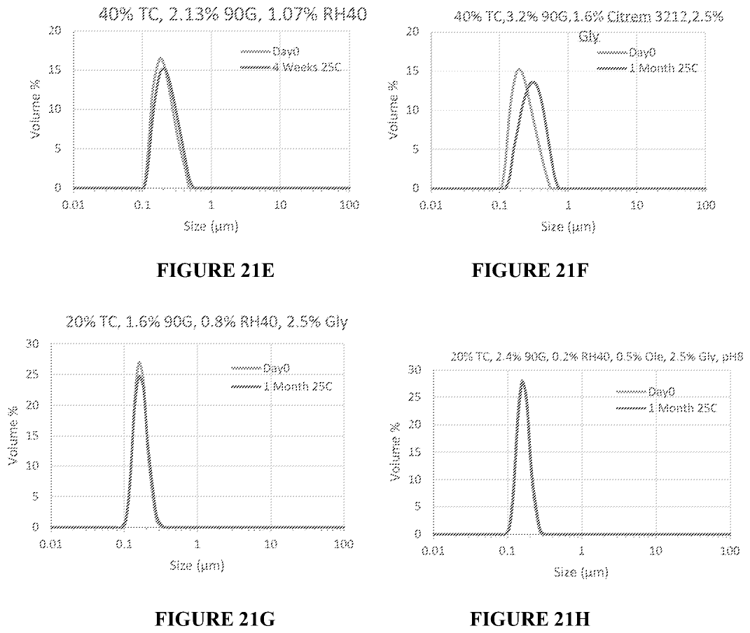

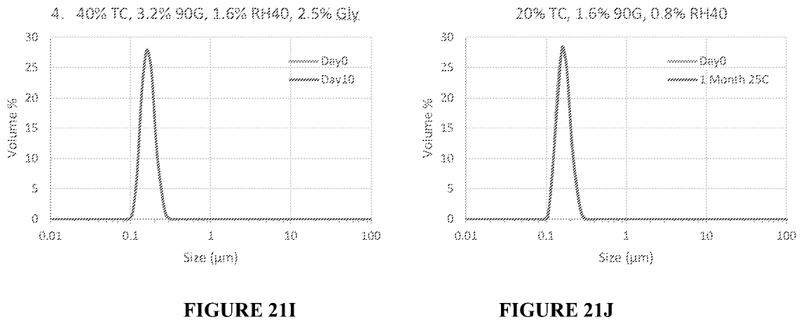

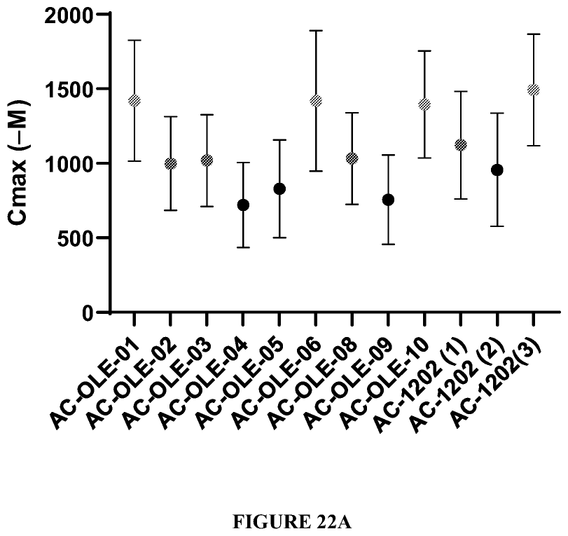

FIGS. 21A-21J illustrate particle size distribution at various time points for lead formulations AC-OLE-1 through AC-OLE-10, as indicated.

### Example 11—pK Study of Lead Formulations

The lead formulations of Example 10 (AC-OLE-1 through AC-OLE-10) were administered to healthy volunteers to investigate pharmacokinetic effects of the liquid formulations of disclosure.

The investigation will include several testing sites and cycles. At east site and in each cycle, several of the lead formulations (up to 4) will be tested on up to 20 healthy volunteers in a partial or full cross-over design. Once each cycle is completed, blood parameters will be analysed.

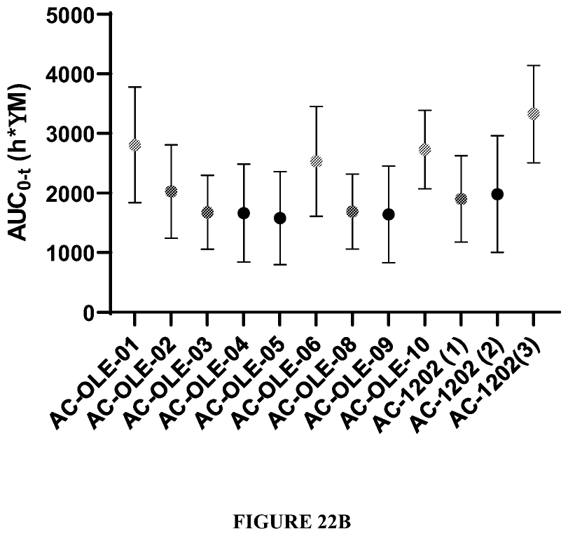

As illustrated in FIG. 22A (Cmax of Total Ketones (BHB+AcA) and FIG. 22B (AUC of Total Ketones (BHB+AcA)) will be calculated as area under the curve (AUC) of total ketones (BHB+AcA), all lead formulations exhibit a total ketone Cmax of over 700 μM, with most over 1000 μM (and generally comparative to prior formulation AC-1202)

All publications and patent applications cited in this specification are herein incorporated by reference as if each individual publication or patent application were specifically, and individually, indicated to be incorporated by reference.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the invention not be limited to the particular embodiment disclosed as the best mode contemplated for carrying out this invention, but that the invention will include all embodiments falling within the scope of the appended claims.

1. A liquid pharmaceutical composition comprising at least about 30% by weight of the total composition of caprylic triglyceride, and one or more emulsion forming excipients present at a concentration sufficient to form an emulsion that is stable for at least one month under ambient conditions.

2. The liquid pharmaceutical composition of claim 1, wherein the one or more emulsion forming excipients are selected from the group consisting of lecithin, hydrogenated castor oils, caprylate esters, sodium oleate, glycerol, citric acid esters of monoglycerides and diglycerides, monoglycerides and diglycerides of fatty acids including Propylene Glycol Monocaprylate, and combinations thereof

3. The liquid pharmaceutical composition of claim 1, wherein the one or more emulsion forming excipients are selected from the group consisting of lecithin, hydrogenated castor oils, caprylate ester emulsifiers, glycerol, and combinations thereof.

4. The liquid pharmaceutical composition of claim 1, wherein the one or more emulsion forming excipients are selected from the group consisting of lecithin, sodium oleate, glycerol, and combinations thereof.

5. The liquid pharmaceutical composition of claim 1, wherein the one or more emulsion forming excipients are selected from the group consisting of Citrem, monoglycerides and diglycerides of fatty acids, and combinations thereof.

6. The liquid pharmaceutical composition of claim 1, wherein the caprylic triglyceride is present in an amount of between about 30% and about 60% by weight of the total composition.

7. The liquid pharmaceutical composition of claim 1, wherein the one or more emulsion forming excipients are present in an amount of between about 1% and about 10% by weight of the total composition, preferably in an amount of between about 1% and about 8% by weight of the total composition.

8. The liquid pharmaceutical composition of claim 1, wherein there are at least two emulsion forming excipients present in the composition, and at least one of the emulsion forming excipients is present in an amount of at least 2.0% by weight of the total composition.

9. The liquid pharmaceutical composition of claim 8, wherein the at least two emulsion forming excipients are present at a 1:1 to 2:1 ratio, relative to one another.

10. The liquid pharmaceutical composition of claim 1, wherein the stable emulsions exhibits an average particle diameter of less than 0.5 μm for at least one month at ambient conditions, preferrable less than 0.3 μm for at least one month at ambient conditions, preferrable less than 0.2 μm for at least one month at ambient conditions.

11. The liquid pharmaceutical composition of claim 1, further comprising an oil soluble flavouring agent.

12. The liquid pharmaceutical compositions of claim 1 for use in a method of treating a disease or disorder associated with reduced cognitive function in a subject in need thereof, the method comprising administering to the subject a liquid pharmaceutical composition in an amount effective to elevate ketone body concentrations in said subject to thereby treat said disease or disorder.

13. The liquid pharmaceutical composition of claim 12, wherein the disease or disorder associated with reduced cognitive function is selected from Alzheimer's disease and Age-Associated Memory Impairment.

14. The liquid pharmaceutical composition of claim 13, further comprising determining if the patient lacks the ApoE4 genotype.

15. The liquid pharmaceutical composition of claim 12, wherein the composition is administered at a dose of about 0.05 g/kg/day to about 10 g/kg/day.
